(19) United States
(12) Patent Application Publication
(10) Pub. No.: US 2024/0277528 A1
(43) Pub. Date: Aug. 22, 2024

(54) ADVANCED EAR ACCESS

(71) Applicant: COCHLEAR LIMITED, Macquarie University, NSW (AU)

(72) Inventors: Wolfram Frederik DUECK, Macquarie University, NSW (AU); Daniel SMYTH, Macquarie University, NSW (AU)

(21) Appl. No.: 18/571,423

(22) PCT Filed: Jun. 16, 2022
(86) PCT No.: PCT/IB2022/055619

§ 371 Date: Dec. 18, 2023

Related U.S. Application Data
(60) Provisional application No. 63/212,582, filed on Jun. 18, 2021.

(51) Int. Cl. A61F 11/20; A61M 31/00

(52) U.S. Cl. CPC A61F 11/20; A61M 31/00; A61M 2210/0662

(57) ABSTRACT

A method, including accessing an ear system of a live human, transporting a material in a first state so that the material comes into contact with a wall of the outer ear, at least permitting the first material to transform to a second state and removing the material in the second state from contact with the wall of the outer ear, wherein the material in the second state retains a memory of a shape of the wall of the outer ear into which the material was in contact when transforming to the second state.

### CROSS-REFERENCE TO RELATED APPLICATIONS

This application claims priority to U.S. Provisional Application No. 63/212,582, entitled ADVANCED EAR ACCESS, filed on Jun. 18, 2021, naming Wolfram Frederik DUECK of Hannover, Germany as an inventor, the entire contents of that application being incorporated herein by reference in its entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In an exemplary embodiment, there is a method, comprising accessing an ear system of a live human, transporting a material in a first state so that the material comes into contact with a wall of the outer ear, at least permitting the first material to transform to a second state, and removing the material in the second state from contact with the wall of the outer ear, wherein the material in the second state retains a memory of a shape of the wall of the outer ear into which the material was in contact when transforming to the second state.

An exemplary embodiment includes an apparatus, comprising a negative model of an inner wall surface of an ear canal of an outer ear unique to a specific person, which model has outer contours to the surface of the actual wall of the ear canal of the specific person.

An exemplary embodiment includes a method, comprising obtaining at least an embryonic three dimensional physical surgical guide or base model for at least an embryonic three dimensional physical surgical guide having surfaces that are variously based on data that is based on spatial locations of surface(s) of an outer ear ear canal and a barrier between a middle ear and an inner ear of a specific human that is alive at the time of obtaining.

An exemplary embodiment includes an apparatus, comprising a hollow medical component and a support structure distinct from the hollow medical component and supporting the hollow medical component, the support structure being configured to interface with an interior of a unique ear canal of a specific human and support the hollow medical component in a fixed trajectory relative to a target in a middle ear of the specific human.

An exemplary embodiment includes an apparatus, comprising a medical catheter having an elongate body extending at least three centimeters and an ear plug distinct from the hollow medical component and supporting the hollow medical component, the ear plug having a surface that is a negative of a unique ear canal of a specific human, wherein the earplug is configured to support the medical catheter in a fixed trajectory relative to a target in a middle ear of the specific human.

### BRIEF DESCRIPTION OF THE DRAWINGS

Embodiments are described below with reference to the attached drawings, in which:

FIG. 1 is a perspective view of an exemplary hearing prosthesis;

FIG. 1A is a perspective view of another exemplary prosthesis;

FIGS. 2A-2H are views of exemplary electrode arrays to which the teachings detailed herein can be applicable;

FIGS. 3A and 3B are side and perspective views of an electrode assembly extended out of an embodiment of an insertion sheath of the insertion tool illustrated in FIG. 2;

FIGS. 4A-4E are simplified side views depicting an exemplary insertion process of the electrode assembly into the cochlea;

FIGS. 5-7 present an exemplary embodiment of an exemplary therapeutic substance delivery system;

FIG. 8 presents an ear system with which embodiments can be used;

FIGS. 9, 10 and 19 present an exemplary embodiment used with the ear system of FIG. 8;

FIGS. 11 and 11A and 12 present exemplary tools;

FIGS. 13-16 present use of the tool of FIG. 12;

FIGS. 17 and 18 present the results of the use of the tool of FIG. 12;

FIG. 19A presents use of the embodiment of FIG. 19;

FIGS. 19B to 19E present exemplary flowcharts for exemplary methods;

FIG. 19F depicts a view of a target;

FIGS. 20 and 21 present an exemplary scanning tool that can be used in some embodiments;

FIG. 22 presents another exemplary embodiment of an ear canal guide;

FIG. 23 presents the embodiment of FIG. 22 in use;

FIGS. 24 and 25 present another exemplary embodiment;

FIGS. 26 and 26 present another exemplary embodiment;

FIG. 28 presents another exemplary embodiment;

FIG. 29 presents another exemplary embodiment; and

FIGS. 30-32 depict exemplary endoscopes used in some embodiments.

### DETAILED DESCRIPTION

Merely for ease of description, the techniques presented herein are described herein with reference by way of background to an illustrative medical device, namely a cochlear implant. However, it is to be appreciated that the techniques presented herein may also be used with a variety of other medical devices that, while providing a wide range of therapeutic benefits to recipients, patients, or other users, may benefit from setting changes based on the location of the medical device. For example, the techniques presented herein may be used to work with various types of prostheses, such as, for example, a vestibular implant and/or a retinal implant, with respect to a particular human being. And with regard to the latter, the techniques presented herein are also described with reference by way of background to another illustrative medical device, namely a retinal implant. The techniques presented herein are also applicable to the technology of vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation, etc.

And while the teachings detailed herein are directed towards accessing the middle ear and/or inner ear, and/or vestibular system, etc., it is noted that any disclosure herein with respect to a hearing system in general, and accessing the middle ear and/or the inner ear in particular, corresponds to a disclosure of an alternate embodiment with respect to accessing a portion of an eye system, as well as accessing or modifying or working with ore replacing a component of a retinal implant/vision implant, such disclosure being made in the interest of textual economy.

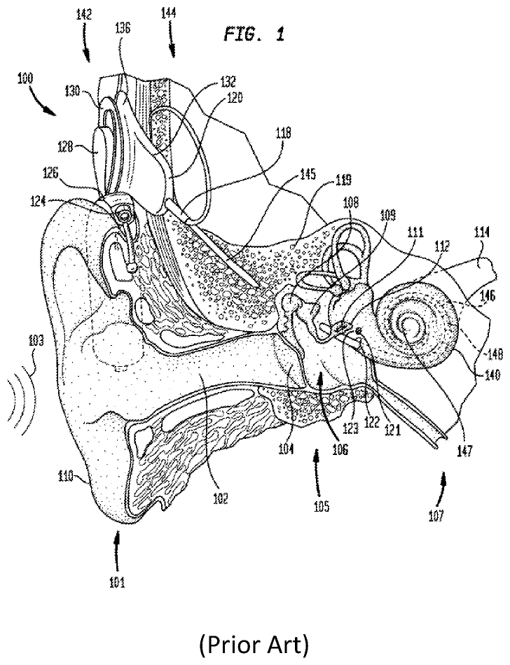

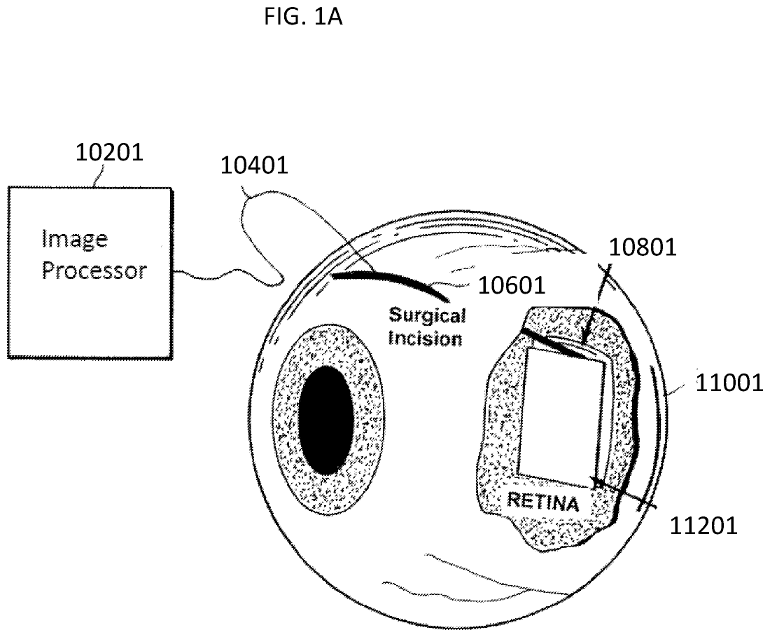

FIG. 1 is a perspective view of an exemplary cochlear implant 100 implanted in a recipient having an outer ear 101, a middle ear 105, and an inner ear 107. In a fully functional ear, outer ear 101 comprises an auricle 110 and an ear canal 102. Acoustic pressure or sound waves 103 are collected by auricle 110 and channeled into and through ear canal 102. Disposed across the distal end of ear canal 102 is a tympanic membrane 104 that vibrates in response to sound waves 103. This vibration is coupled to oval window or fenestra ovalis 112 through the three bones of the middle ear 105, collectively referred to as the ossicles 106, and comprising the malleus 108, the incus 109, and the stapes 111. Ossicles 106 filter and amplify the vibrations delivered by tympanic membrane 104, causing oval window 112 to articulate, or vibrate. This vibration sets up waves of fluid motion of the perilymph within cochlea 140. Such fluid motion, in turn, activates hair cells (not shown) inside the cochlea which in turn causes nerve impulses to be generated which are transferred through spiral ganglion cells (not shown) and auditory nerve 114 to the brain (also not shown) where they are perceived as sound.

The exemplary cochlear implant illustrated in FIG. 1 is a partially-implanted stimulating medical device. Specifically, cochlear implant 100 comprises external components 142 attached to the body of the recipient, and internal or implantable components 144 implanted in the recipient. External components 142 typically comprise one or more sound input elements for detecting sound, such as microphone 124, a sound processor (not shown), and a power source (not shown). Collectively, these components are housed in a behind-the-ear (BTE) device 126 in the example depicted in FIG. 1. External components 142 also include a transmitter unit 128 comprising an external coil 130 of a transcutaneous energy transfer (TET) system. Sound processor 126 processes the output of microphone 124 and generates encoded stimulation data signals which are provided to external coil 130.

Internal components 144 comprise an internal receiver unit 132 including a coil 136 of the TET system, a stimulator unit 120, and an elongate stimulating lead assembly 118. Internal receiver unit 132 and stimulator unit 120 are hermetically sealed within a biocompatible housing commonly referred to as a stimulator/receiver unit. Internal coil 136 of receiver unit 132 receives power and stimulation data from external coil 130. Stimulating lead assembly 118 has a proximal end connected to stimulator unit 120, and extends through mastoid bone 119. Lead assembly 118 has a distal region, referred to as electrode assembly 145, a portion of which is implanted in cochlea 140.

Electrode assembly 145 can be inserted into cochlea 140 via a cochleostomy 122, or through round window 121, oval window 112, promontory 123, or an opening in an apical turn 147 of cochlea 140. Integrated in electrode assembly 145 is an array 146 of longitudinally-aligned and distally extending electrode contacts 148 for stimulating the cochlea by delivering electrical, optical, or some other form of energy. Stimulator unit 120 generates stimulation signals each of which is delivered by a specific electrode contact 148 to cochlea 140, thereby stimulating auditory nerve 114.

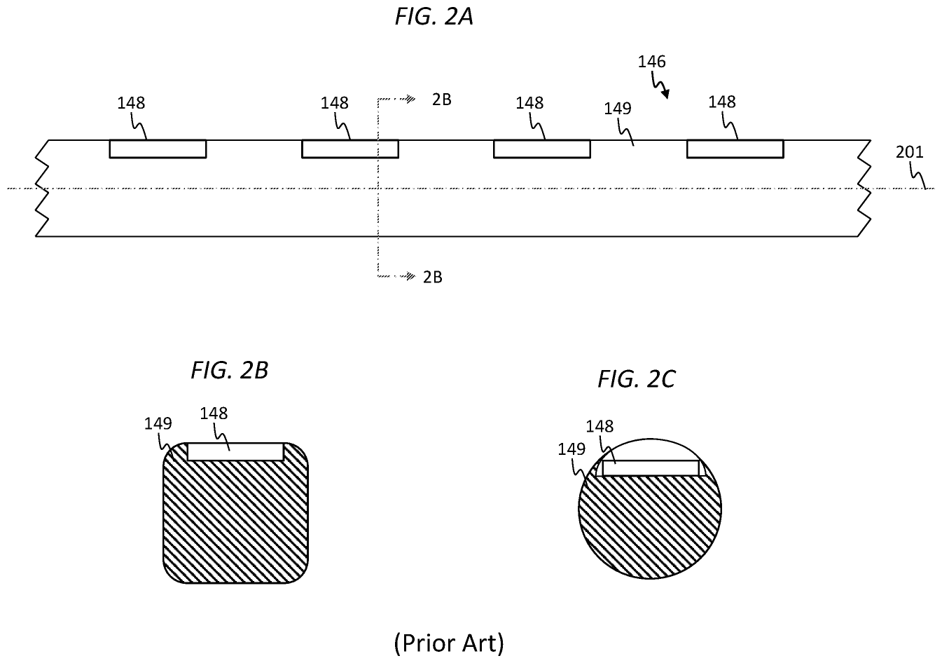

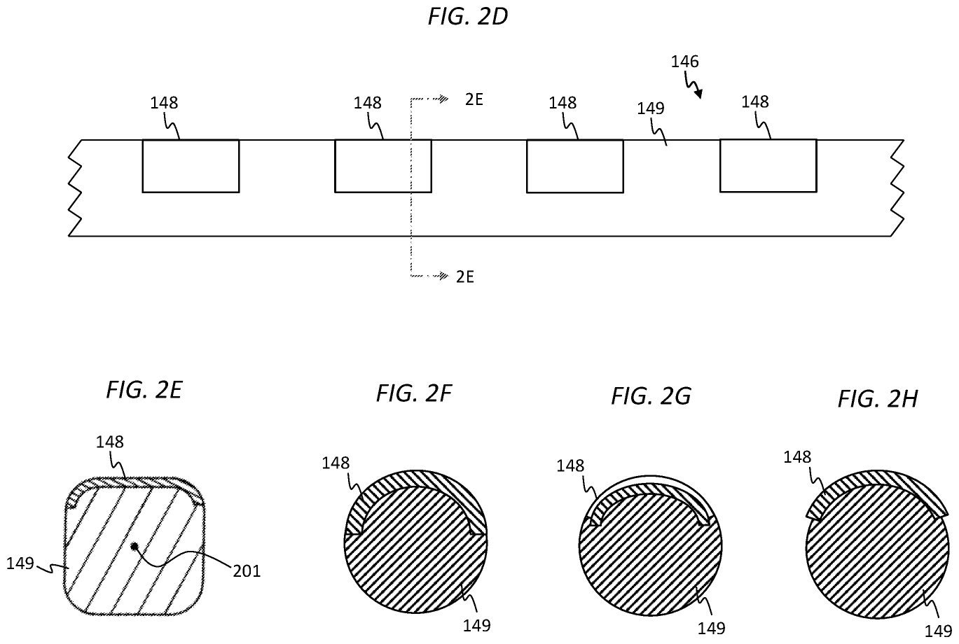

FIG. 2 presents an exemplary embodiment of a neural prosthesis in general, and a retinal prosthesis and an environment of use thereof, in particular, the components of which can be used in whole or in part, in some of the teachings herein. In some embodiments of a retinal prosthesis, a retinal prosthesis sensor-stimulator 10801 is positioned proximate the retina 11001. In an exemplary embodiment, photons entering the eye are absorbed by a microelectronic array of the sensor-stimulator 10801 that is hybridized to a glass piece 11201 containing, for example, an embedded array of microwires. The glass can have a curved surface that conforms to the inner radius of the retina. The sensor-stimulator 108 can include a microelectronic imaging device that can be made of thin silicon containing integrated circuitry that convert the incident photons to an electronic charge.

An image processor 10201 is in signal communication with the sensor-stimulator 10801 via cable 10401 which extends through surgical incision 00601 through the eye wall (although in other embodiments, the image processor 10201 is in wireless communication with the sensor-stimulator 10801). The image processor 10201 processes the input into the sensor-stimulator 10801 and provides control signals back to the sensor-stimulator 10801 so the device can provide processed output to the optic nerve. That said, in an alternate embodiment, the processing is executed by a component proximate with or integrated with the sensor-stimulator 10801. The electric charge resulting from the conversion of the incident photons is converted to a proportional amount of electronic current which is input to a nearby retinal cell layer. The cells fire and a signal is sent to the optic nerve, thus inducing a sight perception.

The retinal prosthesis can include an external device disposed in a Behind-The-Ear (BTE) unit or in a pair of eyeglasses, or any other type of component that can have utilitarian value. The retinal prosthesis can include an external light/image capture device (e.g., located in/on a BTE device or a pair of glasses, etc.), while, as noted above, in some embodiments, the sensor-stimulator 10801 captures light/images, which sensor-stimulator is implanted in the recipient.

FIG. 2A depicts a conceptual side view of a portion of electrode array 146, depicting four electrode contacts 148 evenly spaced along a longitudinal axis of the electrode array 146. It is noted that in some alternate embodiments, the electrode is not evenly spaced. FIG. 2B depicts a conceptual cross-sectional view through one of the electrode contacts 148, which also depicts the carrier 149 of the electrode contact 148. In an exemplary embodiment, the carrier 149 is made of silicone. Not depicted in the figures are electrical leads and stiffener components that are sometimes embedded in the carrier 149. The embodiment of FIG. 2B represents an electrode array 146 that has a generally rectangular cross-section. FIG. 2C depicts an alternate embodiment where the electrode array 146 has a generally circular cross-section. It is also noted that in some exemplary embodiments, the cross-section is oval shaped. Thus, the embodiment of FIGS. 2A-2C is a species of the genus of an electrode array having a generally continuously curving cross-section. Any electrode array of any cross-section or any configuration can be utilized with the teachings detailed herein.

The electrode contacts 148 depicted in FIGS. 2A-2C are so-called flat contacts. In this regard, the surface of the electrode contact that faces the wall of the cochlea/the faces away from the longitudinal axis of the electrode array 146 is flat. Conversely, as seen in FIGS. 2D-2H, in some alternate embodiments, the electrode contacts 148 are so-called half band electrodes. In some exemplary embodiments, a band of contact material is “smashed” or otherwise compressed into a “half band,” as seen in the figures. It is noted that by “half band,” this does not mean that the electrode contact must necessarily span half of the outside diameter of the electrode array, as is the case in FIGS. 2G and 2H. The term is directed towards the configuration of the electrode itself as that term has meaning in the art. Any electrode contact that can have utilitarian value according to the teachings detailed herein can be utilized in at least some exemplary embodiments.

As can be seen from FIGS. 2A-2H, the positioning of the electrode contacts relative to the carrier 149 can vary with respect to alignment of the outer surface of the carrier with the outer surface of the contact. For example, FIGS. 2A, 2E, and 2F depict the outer surface of the contacts 148 as being flush with the outer surface of the carrier 149. Conversely, FIGS. 2C and 2G depict the contact 148 as being recessed with respect to the outer surface of the carrier 149, while FIG. 2H depicts the contact 148 as being proud relative to the outer surface of the contact 149. It is noted that these various features are not limited to the specific contact geometry and/or the specific carrier geometry depicted in the figures, and that one or more features of one exemplary embodiment can be combined with one or more features of another exemplary embodiment. For example, while FIG. 2H depicts a half band contact as being proud of the carrier 149 having a generally circular cross-section, a flat electrode such as that depicted in FIG. 2A can be proud of the carrier as well.

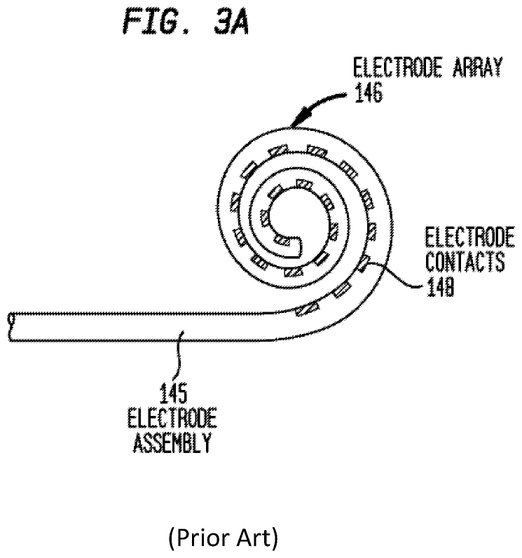

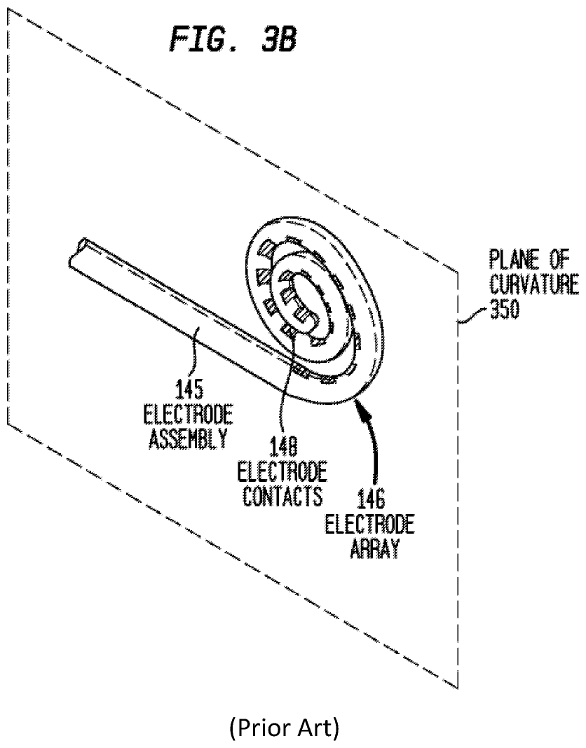

FIGS. 3A and 3B are side and perspective views, respectively, of representative electrode assembly 145. As noted, electrode assembly 145 comprises an electrode array 146 of electrode contacts 148. Electrode assembly 145 is configured to place electrode contacts 148 in close proximity to the ganglion cells in the modiolus. Such an electrode assembly, commonly referred to as a perimodiolar electrode assembly, is manufactured in a curved configuration as depicted in FIGS. 3A and 3B. When free of the restraint of a stylet or insertion guide tube, electrode assembly 145 takes on a curved configuration due to it being manufactured with a bias to curve, so that it is able to conform to the curved interior of cochlea 140. As shown in FIG. 3B, when not in cochlea 140, electrode assembly 145 generally resides in a plane 350 as it returns to its curved configuration. That said, it is noted that the teachings detailed herein and/or variations thereof can be applicable to a so-called straight electrode array, which electrode array does not curl after being free of a stylet or insertion guide tube etc., but instead remains straight. It is noted that when in the cochlea, the electrode assembly 145 takes on a conical shape with respect to plane 350 in that it can be described as winding upward away from the plane 350 about an axis normal thereto, owing to the shape of the cochlea (more on this below).

The perimodiolar electrode assembly 145 of FIGS. 3A and 3B is pre-curved in a direction that results in electrode contacts 148 being located on the interior of the curved assembly, as this causes the electrode contacts to face the modiolus when the electrode assembly is implanted in or adjacent to cochlea 140.

It is also noted that while the embodiments of FIGS. 2A-3B have been presented in terms of a so-called non-tapered electrode array (where the cross-sections of the array on a plane normal to the longitudinal axis at various locations along the longitudinal axis (e.g., in between each electrode (or a majority of the electrodes), in the middle of each electrode (or a majority of the electrodes) etc.) have generally the same cross-sectional area and shape), in an alternate embodiment, the teachings detailed herein can be applicable to a so-called tapered electrode, where the cross-sectional areas on planes taken normal to the longitudinal axis decrease with location towards the distal end of the electrode array.

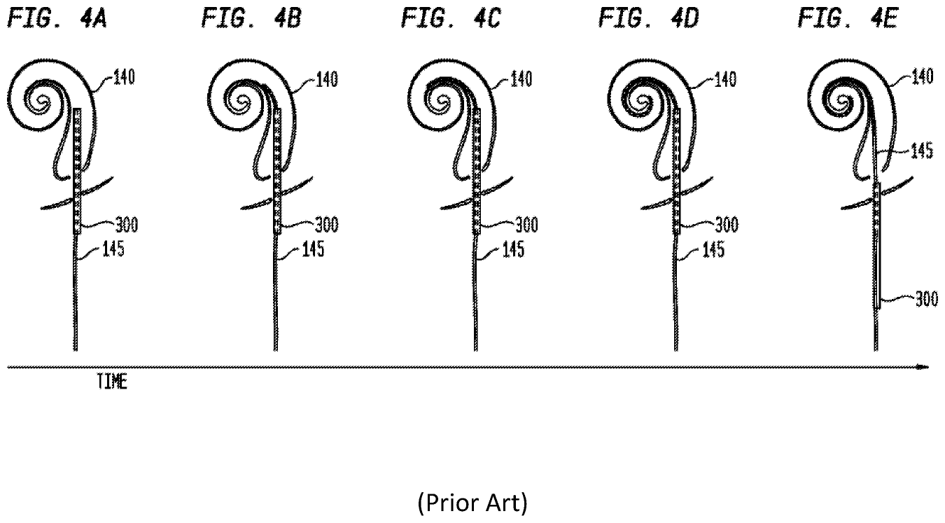

FIGS. 4A-4E depict an exemplary insertion regime of an electrode assembly according to an exemplary embodiment. As shown in FIG. 4A, the combined arrangement of an insertion guide tube 300 and electrode assembly 145 is substantially straight. This is due in part to the rigidity of insertion guide tube 300 relative to the bias force applied to the interior wall of the guide tube by pre-curved electrode assembly 145.

As noted, in some embodiments, the electrode assembly 145 is biased to curl and will do so in the absence of forces applied thereto to maintain the straightness. That is, electrode assembly 145 has a memory that causes it to adopt a curved configuration in the absence of external forces. As a result, when electrode assembly 145 is retained in a straight orientation in guide tube 300, the guide tube prevents the electrode assembly from returning to its pre-curved configuration. In the embodiment configured to be implanted in scala tympani of the cochlea, electrode assembly 145 is pre-curved to have a radius of curvature that approximates and/or is less than the curvature of medial side of the scala tympani of the cochlea. Such embodiments of the electrode assembly are referred to as a perimodiolar electrode assembly, and this position within cochlea 140 is commonly referred to as the perimodiolar position. In some embodiments, placing electrode contacts in the perimodiolar position provides utility with respect to the specificity of electrical stimulation, and can reduce the requisite current levels thereby reducing power consumption.

As shown in FIGS. 4B-4D, electrode assembly 145 may be continually advanced through insertion guide tube 300 while the insertion sheath is maintained in a substantially stationary position. This causes the distal end of electrode assembly 145 to extend from the distal end of insertion guide tube 300. As it does so, the illustrative embodiment of electrode assembly 145 bends or curves to attain a perimodiolar position, as shown in FIGS. 4B-4D, owing to its bias (memory) to curve. Once electrode assembly 145 is located at the desired depth in the scala tympani, insertion guide tube 300 is removed from cochlea 140 while electrode assembly 145 is maintained in a stationary position. This is illustrated in FIG. 4E.

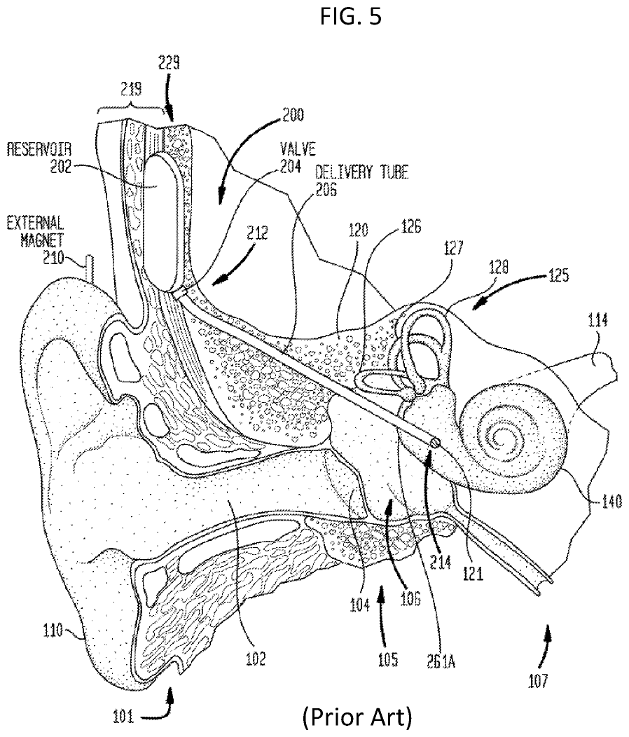

FIG. 5 depicts an exemplary drug delivery device, the details of which will be provided below. As shown in FIG. 5, semicircular canals 125 are three half-circular, interconnected tubes located adjacent cochlea 140. Vestibule 129 provides fluid communication between semicircular canals 125 and cochlea 140. The three canals are the horizontal semicircular canal 126, the posterior semicircular canal 127, and the superior semicircular canal 128. The canals 126, 127, and 128 are aligned approximately orthogonally to one another. Specifically, horizontal canal 126 is aligned roughly horizontally in the head, while the superior 128 and posterior canals 127 are aligned roughly at a 45 degree angle to a vertical through the center of the individual's head.

It can be utilitarian to have a prompt and/or extended delivery solution for use in the delivery of treatment substances to a target location of a recipient. In general, extended treatment substance delivery refers to the delivery of treatment substances over a period of time (e.g., continuously, periodically, etc.). The extended delivery may be activated during or after surgery and can be extended as long as is needed. The period of time may not immediately follow the initial implantation of the auditory prosthesis. Embodiments of the teachings herein can facilitate extended delivery of treatment substances, as well as facilitating prompt delivery of such substances.

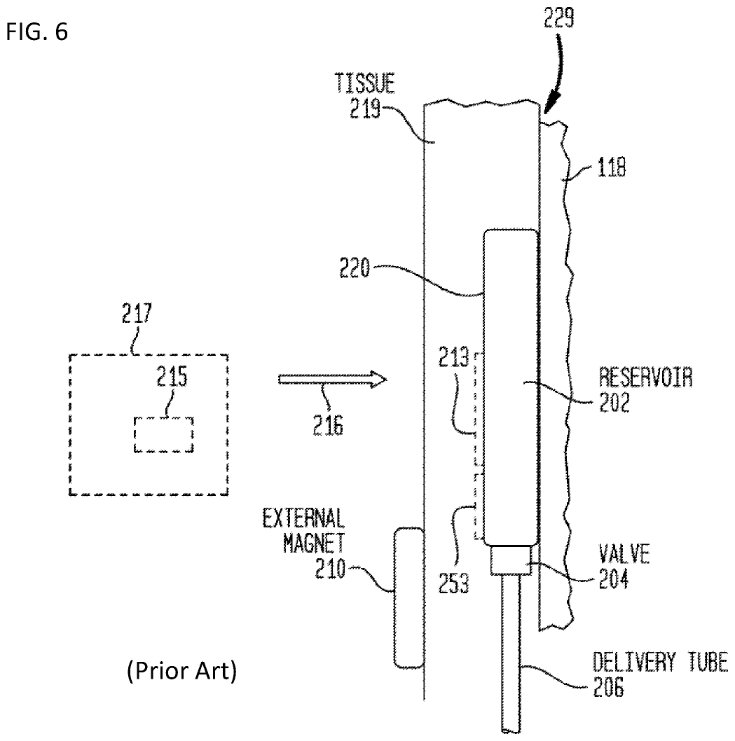

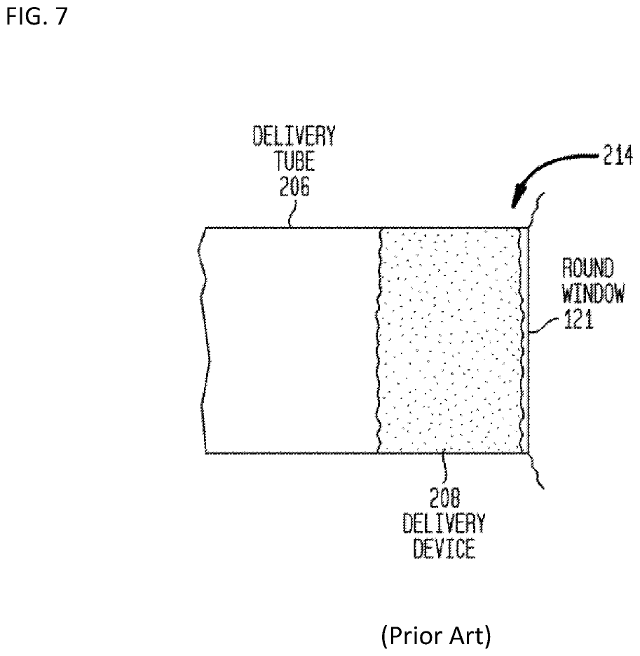

FIG. 5 illustrates an implantable delivery system 200 that can be utilized with the teachings detailed herein, and otherwise modified as detailed by way of example below. The delivery system has a passive actuation mechanism. However, it is noted that the delivery system 200 can also or instead have an active actuation system. The delivery system 200 is sometimes referred to herein as an inner ear delivery system because it is configured to deliver treatment substances to the recipient's inner ear (e.g., the target location is the interior of the recipient's cochlea 140 (in other embodiments, this could be provided to the semi-circular canals). FIG. 6 illustrates a first portion of the delivery system 200, while FIG. 7 is a cross-sectional view of a second portion of the delivery system 200.

Delivery system 200 of FIGS. 5-7 comprises a reservoir 202, a valve 204, a delivery tube 206, and a delivery device 208 (FIG. 7). The delivery system 200 can include, or operate with, an external magnet 210. For ease of illustration, the delivery system 200 is shown separate from any implantable auditory prostheses. However, it is to be appreciated that the delivery system 200, and any of the other delivery systems detailed herein and/or variations thereof, could be used with, for example, cochlear implants, such as that presented in FIG. 1, direct acoustic stimulators, middle ear implants, bone conduction devices, etc. The implantable components (e.g., reservoir, valve, delivery tube, etc.) of delivery system 200 (or any other delivery system detailed herein) could be separate from or integrated with the other components of the implantable auditory prosthesis.

The reservoir 202 is positioned within the recipient underneath a portion of the recipient's skin/muscle/fat, collectively referred to herein as tissue 219. The reservoir 202 may be positioned between layers of the recipient's tissue 219 or may be adjacent to a subcutaneous outer surface 229 of the recipient's skull. For example, the reservoir 202 may be positioned in a surgically created pocket at the outer surface 229 (i.e., adjacent to a superior portion 118 of the temporal bone 115).

The reservoir 202 is, prior to or after implantation, at least partially filled with a treatment substance for delivery to the inner ear 107 of the recipient. The treatment substance may be, for example, in a liquid form, a gel form, and/or comprise nanoparticles or pellets. In certain arrangements, the treatment substance may initially be in a crystalline/solid form that is subsequently dissolved. For example, a reservoir could include two chambers, one that comprises a fluid (e.g., artificial perilymph or saline) and one that comprises the crystalline/solid treatment substance. The fluid may be mixed with the crystalline/solid treatment substance to form a fluid or gel treatment substance that may be subsequently delivered to the recipient.

The reservoir 202 includes a needle port (not shown) so that the reservoir 202 can be refilled via a needle injection through the skin. The reservoir 202 may be explanted and replaced with another reservoir that is, prior to or after implantation, at least partially filled with a treatment substance. The reservoir 202 may have a preformed shape and the reservoir is implanted in this shape. The reservoir 202 may have a first shape that facilitates implantation and a second shape for use in delivering treatment substances to the recipient. For example, the reservoir 202 may have a rolled or substantially flat initial shape that facilitates implantation. The reservoir 202 may then be configured to expand after implantation. Such may be used, for example, to insert the reservoir through a tympanostomy into the middle ear or ear canal, through an opening in the inner ear, or to facilitate other minimally invasive insertions.

The delivery tube 206 includes a proximal end 212 and a distal end 214. The proximal end 212 of the delivery tube 206 is fluidically coupled to the reservoir 202 via the valve 204. As shown in FIG. 7, the distal end 214 of the delivery tube 206 is fluidically coupled to the recipient's round window 121. A delivery device 208 disposed within the distal end 214 of the delivery tube 206 is positioned abutting the round window 121. As described further below, the delivery tube 206 may be secured within the recipient so that the distal end 214 remains located adjacent to the round window 121.

FIGS. 5-7 illustrate a system that utilizes utilize a passive actuation mechanism to produce a pumping action to transfer a treatment substance from the reservoir 202 to the delivery device 208 at the distal end 214 of the delivery tube 206. More specifically, in this system, the reservoir 202 is compressible in response to an external force 216. That is, at least one part or portion of the reservoir 202, such as wall 220 or a portion thereof, is formed from a resiliently flexible material that is configured to deform in response to application of the external force 216. In some implementations of the system of FIG. 5, positioning of the reservoir 202 adjacent the superior portion of the mastoid provides a rigid surface that counters the external force 216. As a result, a pressure change occurs in the reservoir 202 so as to propel (push) a portion of the treatment substance out of the reservoir through valve 204.

FIGS. 5 and 6 illustrate a specific arrangement in which the reservoir 202 includes a resiliently flexible wall 220. It is to be appreciated that the reservoir 202 can be formed from various resiliently flexible parts and rigid parts. It is also to be appreciated that the reservoir 202 may have a variety of shapes and sizes (e.g., cylindrical, square, rectangular, etc.) or other configurations. For example, the reservoir 202 could further include a spring mounted base that maintains a pressure in the reservoir 202 until the reservoir is substantially empty.

Other mechanisms for maintaining a pressure in the reservoir may be used in other arrangements.

The external force 216 is applied manually using, for example, a user's finger. The user (e.g., recipient, clinician, caregiver, etc.) may press on the tissue 219 adjacent to the reservoir 202 to create the external force 216. A single finger press may be sufficient to propel the treatment substance through valve 204. In some instances, a multiple finger presses may be used to create a pumping action that propels the treatment substance from the reservoir 202.

The external force 216 is applied through a semi-manual method that uses an external actuator 217 (FIG. 2B). That is, the external actuator 217 may be pressed onto the soft tissue 219 under which the reservoir 202 is located. The movement (e.g., oscillation/vibration) of the actuator 217 deforms the reservoir 202 to create the pumping action that propels the treatment substance out of the reservoir.

Internal and/or external magnets and/or magnetic materials may be used in the arrangements of FIGS. 5 and 6 to ensure that the actuator 217 applies force at an optimal location of the reservoir 202. For example, the reservoir 202 may include a magnetic positioning member 213 located at or near an optimal location for application of an external force from the actuator 217. The actuator 217 may include a magnet 215 configured to magnetically mate with the magnetic positioning member 213. As such, when actuator 217 is properly positioned, the magnet 215 will mate with the magnetic positioning member 213 and the force from the actuator 217 will be applied at the optimal location.

A remote control, remotely placed actuator (subcutaneous or otherwise) may be alternatively used. For example, in a further arrangement, the implant includes implanted electronics 253 (shown using dotted lines in FIG. 6). These implanted electronics 253 may be configured to, for example, control the valve 204 and/or include an actuation mechanism that can force treatment substance from the reservoir 202. The implanted electronics 253 may be powered and/or controlled through a transcutaneous link (e.g., RF link). As such, the implanted electronics 253 may include or be electrically connected to an RF coil, receiver/transceiver unit, etc.

The implanted electronics 253 may include or be connected to a sensor that is used, at least in part, to assist in control of delivery of the treatment substance to the recipient. For example, a sensor (e.g., a temperature sensor, a sensor to detect infection or bacteria growth, etc.) may provide indications of when a treatment substance should be delivered and/or when delivery should be ceased for a period of time. A sensor may also be configured to determine an impact of the treatment substance on the recipient (e.g., evaluate effectiveness of the treatment substance).

As noted, the treatment substance (sometimes herein referred to as therapeutic substance) is released from the reservoir 202 through the valve 204. The valve 204 may be a check valve (one-way valve) that allows the treatment substance to pass therethrough in one direction only. This assures that released treatment substances do not back-flow into the reservoir 202. The valve 204 is a valve that is configured to open in response to the pressure change in the reservoir 202 (e.g., a ball check valve, diaphragm check valve, swing check valve or tilting disc check valve, etc.). The valve 204 may be a stop-check valve that includes an override control to stop flow regardless of flow direction or pressure. That is, in addition to closing in response to backflow or insufficient forward pressure (as in a normal check valve), a stop-check value can also be deliberately opened or shut by an external mechanism, thereby preventing any flow regardless of forward pressure. The valve 204 may be a stop-check value that is controlled by an external electric or magnetic field generated by, for example, the external magnet 210, an electromagnet, etc. In the system of FIGS. 5 and 6, the valve is responsive to a magnetic field generated by external magnet 210. As such, the valve 204 will temporarily open when the external magnet 210 is positioned in proximity to the valve 204 and will close when the external magnet 210 is removed from the proximity of the valve 204. Variable magnet strengths of external magnets may be used to control the dosage of the treatment substance. Additionally, an electromagnet may be used in place of the external magnet 210.

The use of a stop-check valve can prevent unintended dosing of the treatment substance when, for example, an accidental external force acts on the reservoir 202. The reservoir 202 is formed such that an increase in pressure of the reservoir 202 without an accompanying treatment substance release will not damage (i.e., rupture) the reservoir.

The use of a magnetically activated stop-check valve is merely exemplary and that other types of valves may be used. For example, the valve 204 may be actuated (i.e., opened) in response to an electrical signal (e.g., piezoelectric valve). The electrical signal may be received from a portion of an auditory prosthesis (not shown) that is implanted with the delivery system 200 or the electrical signal may be received from an external device (e.g., an RF actuation signal received from an external sound processor, remote control, etc.). In some instances, manually applied (e.g., finger) force be also able to open the valve 204.

Once the treatment substance is released through valve 204, the treatment substance flows through the delivery tube 206 to the delivery device 208. The delivery device 208 operates as a transfer mechanism to transfer the treatment substance from the delivery tube 206 to the round window 121. The treatment substance may then enter the cochlea 140 through the round window 121 (e.g., via osmosis). The delivery device 208 may be, for example, a wick, a sponge, permeating gel (e.g., hydrogel), etc.

The reservoir 202 may include a notification mechanism that transmits a signal or notification indicating that the reservoir 202 is substantially empty and/or needs refilled. For example, one or more electrode contacts (not shown) may be present and become electrically connected when the reservoir is substantially empty. Electronic components associated with or connected to the reservoir 202 may accordingly transmit a signal indicating that reservoir needs filled or replaced.

FIGS. 5-7 illustrate a specific example in which the round window 121 is the target location. As noted above, the round window 121 is an exemplary target location and other target locations are possible. FIGS. 5-7 also illustrate that the reservoir 202 is positioned adjacent to the outer surface 229 of the recipient's skull so that an external force may be used to propel the treatment substance from the reservoir.

While the features of FIGS. 5-7 are directed to an implantable therapeutic substance delivery system of the prior art, with an implanted system, embodiments disclosed herein are directed towards medical tools and methods utilized by surgeons or other healthcare professionals, and, in some embodiments, by recipients or lay people (more on this below) to provide therapeutic substances to the cochlea, and, to tools and methods that provide access to the cochlea (in the first instance or on a repeated or subsequent to the first instance basis), and tools that enable more easy access to a barrier between the middle ear in the inner ear in general, and thus the cochlea in particular. That is, in contrast to the embodiment of FIG. 5, these teachings are directed towards a non-implantable device, although it is noted that in some embodiments, there may be an implanted component involved with the methods, and these teachings can be utilized with an implantable device, such as a port that leads to the cochlea. In a broad sense, the teachings below are directed to tools, not implants, in the sense that one does not implant a tool, but one may utilize a tool in conjunction with an implant. It is noted that some embodiments can use one or more of the aspects of the embodiment of FIG. 5 to enable the teachings below, and otherwise the aspects of the embodiment of FIG. 5 provide teachings that can be adapted for use with the teachings below.

We note also that in at least some exemplary embodiments, the teachings detailed herein can be utilized to implants prostheses components into the cochlea or in the middle ear, or otherwise attached such components of the middle ear. In this regard, in an exemplary embodiment a cochlear implant electrode array can be delivered into a cochlea utilizing a cannula guide that extends through the through bore of the custom ear canal guide to the cochleostomy.

It is noted that in some exemplary embodiments, the therapeutic substance can be delivered to other portions of the ear system, such as by way of example only and not by way of limitation, the semicircular canals, and the tools detailed herein can be utilized to reach such or otherwise access such. Embodiments include the utilization of the techniques and/or tools etc., herein to reach and in some embodiments pierce or otherwise incise tissue of the semicircular canals, and provide therapeutic substance thereto. It is noted that any disclosure herein referencing accessing and/or providing therapeutic substance to the ducts of the cochlea corresponds to an alternate disclosure of an alternate exemplary embodiment of accessing and/or providing therapeutics to the semicircular canals, providing that the art enables such, unless otherwise noted.

Note also that while the various teachings detailed herein are directed towards piercing or otherwise incising through or passing through an existing opening in the tympanic membrane and/or the round window membrane, in an exemplary embodiment, the teachings detailed herein also provide an arrangement that can enable incising through so-called false membranes as well as the true membrane.

At least some embodiments can enable a user to access the far portions of the middle ear (far in terms of relation to the tympanic membrane or the outer ear, for example), such as the barrier between the middle ear and the inner ear, and the inner ear itself, without having a visual of those access locations. In an exemplary embodiment, the teachings detailed herein enable a user to conduct minimally invasive surgery with respect to the far portions of the middle ear and or the inner ear with visualization limited to only the outer ear, at least with respect to some actions (it can be all actions), such as, for example, the action of attaching an artificial component to the barrier between the middle ear and the inner ear and/or drilling through the barrier to establish the cochleostomy. Thus, embodiments include methods that include acting on such enablement.

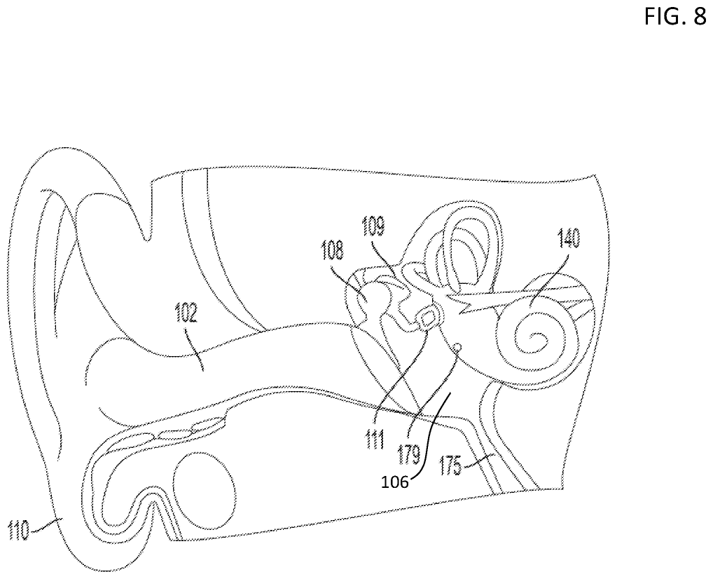

FIG. 8 presents a cross-section of an inner ear of a human without any artificial components therein. This can be considered a starting point for the methods and apparatuses disclosed herein. That said, it is noted that in at least some exemplary embodiments, the tympanic membrane 104 can have a grommet located therein, such as a grommet that is utilized to relieve middle ear pressure and/or a grommet that is utilized to facilitate access to the middle ear. This too can be a baseline for the teachings detailed herein. In an exemplary embodiment, the inner diameter of the grommet utilized in at least some exemplary embodiments can be 0.2, 0.3, 0.4, 0.5, 0.6, 0.7, 0.8, 0.9, 1, 1.25, 1.5, 1.75, 2, 2.25, 2.5, 2.75 3, 3.5, or 4 mm or more, or any value range of values therebetween in 0.01 mm increments, and accordingly, in at least some exemplary embodiments, the components according to the teachings detailed herein that are to be fit through the inner diameter of the grommet (opening) are sized and dimensioned to have diameters that are smaller than the aforementioned inner diameter.

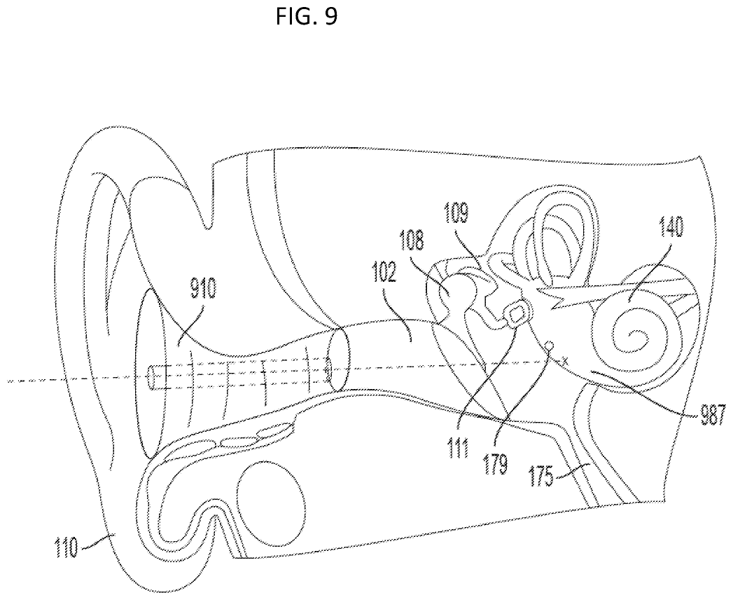

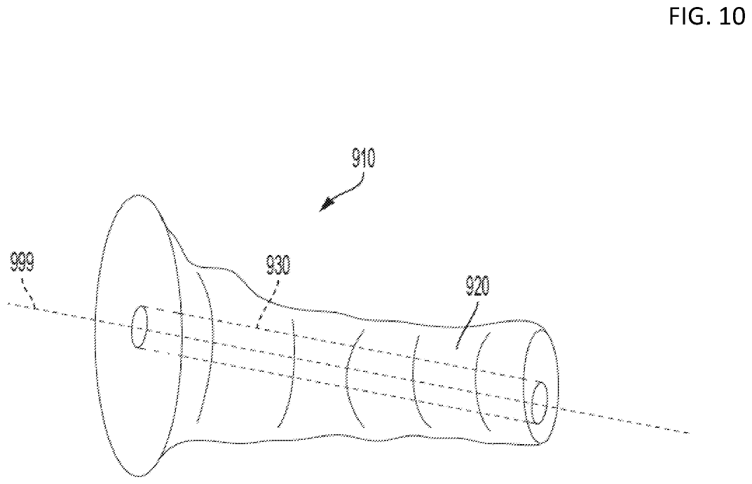

With the above in mind, FIGS. 9 and 10 present an exemplary custom ear canal guide 910 in the form of an ear canal club with an insertion guide for drilling into a barrier between a middle ear and an inner ear, specifically identified as location 987 below the round window niche 179 (where below is based on the fact that the oval window is located above the round window niche with respect to the spatial geometry shown in FIG. 9—it is noted that other embodiments can utilize the teachings detailed herein to drill through the barrier at other locations, such as the barrier between the oval window in the round window niche for example) or otherwise accessing that location (embodiments are limited to drilling as will be described below).

FIG. 10 presents a body 920 that has an outer contour that is custom to an ear canal of a specific person. As seen, a through bore 930 extends from the distal end of the custom ear canal guide 910 to the proximal end of the custom ear canal guide 910. The through bore establishes a longitudinal axis 999 therethrough. The longitudinal axis 999 is straight and is of a sufficient distance that, in combination with a properly sized drill bit or termination (e.g., the outer diameter is large enough, relative to the diameter of the through bore 930 (so there is no wobble or tilting (or at least not enough that would result in a “miss” with respect to the target 987 relative to the distal end of the drill bit or termination, at least not unless there is intentional bending moments placed on the drill bit or termination)), will result in a trajectory thereof extending along the axis 909 of FIG. 9 (where axis 999 is coaxial and parallel with axis 909).

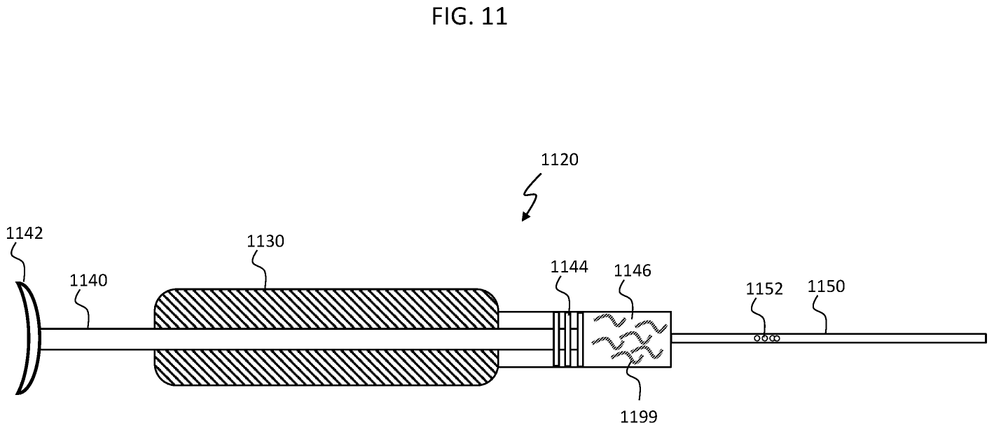

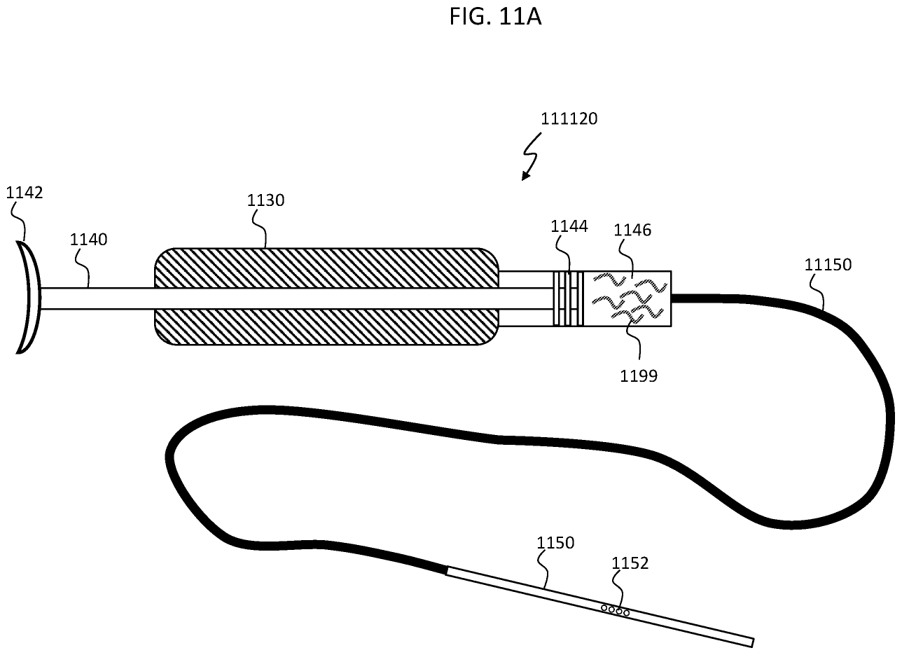

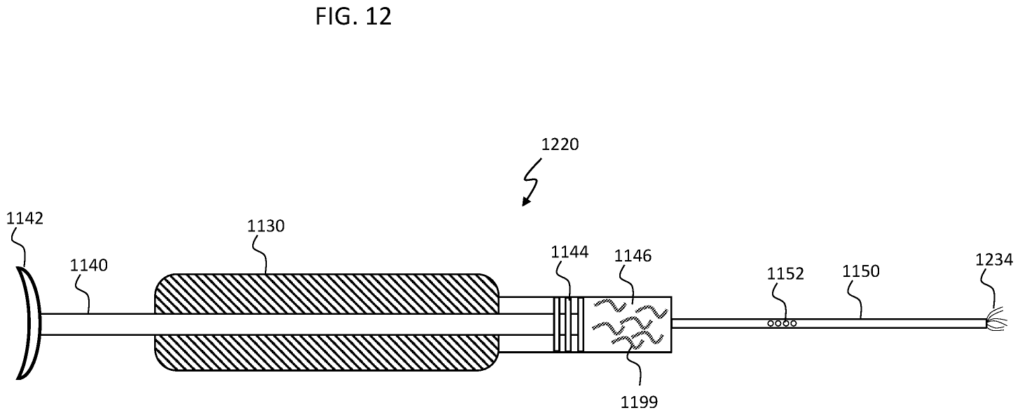

The features of this embodiment will be described in greater detail below, and will initially be described based on a method of manufacture or otherwise establishment of the custom ear canal guide 910. In this regard, FIG. 11 presents an exemplary device 1120 for establishing an embryonic custom ear canal guide. Here, the device 1120 includes a handle 1130 that is ergonomically designed to interface in a utilitarian manner with a human hand of human factors engineering proportions likely to correspond to that of a healthcare professional utilizing the device 1120. Extending to the handle is a rod 1140 that is connected to a reaction surface 1142, which can be a piece of metal or plastic that is curved to interface with a human factors engineering expected thumb of the healthcare professional, while in other embodiments, element 1142 corresponds to a handle that can be gripped in the other hand. The rod 1140 is connected to plunger 1144 which is established by three disks that are sufficiently flexible so that they establish a seal within the cylinder 1199 extending from the handle 1130, but rigid enough that they will push the uncured material 1146 out of the cylinder 1199 and into the termination 1150. In this regard, termination 1150 can be a hollow tube made of stainless steel or titanium or some other material. Termination 1150 includes one or more holes 1152 located along the longitudinal axis thereof. Termination 1150 can optionally also include an opening at the end of the termination. In an exemplary embodiment, device 1120 (or device 1220 below) can be a double barrel device that has to barrels or two compartments each containing a separate material that when separated or otherwise not in contact with each other, these materials are maintained and respective first states (which can be the same) but when combined, these different materials transform into a collective second state, such as a harder state, after a certain period of time. And in this regard, in an exemplary embodiment, the two barrels can be in fluid communication with the termination 1150, where they are mixed, or can be in fluid communication with a “mixing chamber” or some other chamber downstream of the respective barrels where the materials come into contact with each other and then flow into the termination and then out into the ear canal accordingly. Because the two separate (different) materials are in contact with each other, a reaction takes place which transforms the material to the second state. This can have utilitarian value with respect to the utilization of transition materials that come into parts such as a first part A, and a second part B, which parts can be contained in the separate barrels or chambers, and thus in their stable first state(s), and then when it comes time to utilize such, when the two materials come into contact each other, they transition to the second state. In this regard, this can be analogous to, for example, a two part epoxy, with the packaging keeps the two components in a liquid state in separate tubes until the moment they are mixed and applied, where once mixed, they cure into the harder material rather quickly, but typically within timescale that still permits the application of the material, such as, here, for example, where even after the materials are mixed, there still will be enough time to transport the materials down the termination and into the ear canal before the curing takes place at least to a level where hardening exists that prevents further flow.

In an exemplary embodiment, a two component silicone can be utilized, where once the components are mixed or otherwise brought into contact with each other, the material transforms into the second state.

And it is noted that it is possible that in some embodiments, tool 1120 might be a bit cumbersome to hold at the desired trajectory for the amount of time needed to allow the material to cure or otherwise transform from the first state to the second state. Accordingly, in at least some exemplary embodiments, the termination 1150 is detachable from the cylinder 1146 or a portion of the cylinder 1146 is detachable from the remainder of the tool, etc., so that after a sufficient amount of the material has been delivered to the desired areas, the heavier parts of the tool or otherwise the parts that create bending moments that must be restrained against can be removed so that the torque on the termination 1150 is reduced if not eliminated.

Moreover, FIG. 11A presents another exemplary embodiment of the tool 111120 that includes a hose 11150 that is configured to channel the material 11992 the termination 1150. The hose 11150 is flexible. Thus, a surgeon or other healthcare professional can concentrate on the orientation and trajectory of the termination 1150 while the remainder of the tool can lay on a table or another healthcare professional can hold the rest of the tool. This also can reduce the wanes or mass of the portions that are interfacing with the ear system and otherwise reduce the moment on the termination 1150. And in an exemplary embodiment, after a sufficient amount of the material has been directed to the desired locations, the termination 1150 can be removed from the hose 11150. In this regard, the hose 11150 is releasably coupled to the termination 1150.

In an exemplary embodiment, the material can be at least part alginate. Material that is utilized for ear impressions in the United States of America as of Jun. 15, 2021, can also be utilized in some embodiments, including material that is approved by the United States FDA or otherwise by pertinent medical boards in the United States of America on that date.

In an exemplary embodiment, hydrocolloids can be utilized. In an exemplary embodiment, alginate and agar-agar and synthetic elastomers including Polysulphides, Polyether, condensation silicone can be used (one or more or all of these. Also, in an exemplary embodiment, silicone such as polyvinyl siloxane (PVS), polyurethanes, methyl methacrylate polymer and monomer (powder and liquid) can be utilized. Light body impression materials that have utilitarian value vis-à-vis their injectability (low viscosity) can be utilized.

In an exemplary embodiment, the material, in the second state or otherwise the “harder” state, and have a shore harness that is greater than, less than and/or equal to 20 to 80 Shore A Rennie value or range of values therebetween in 1 Shore A increments.

FIG. 12 presents an exemplary embodiment of the hand tool of FIG. 11, except here, tool 1220 includes elements 1234 (these can be electrodes, or soft and/or flexible antennas—and the two are not mutually exclusive) located at the end of the termination. This can have utilitarian value with respect to providing feedback to the user as to whether or not the end of the termination is proximate the barrier between the middle ear and the inner ear. In an exemplary embodiment, the elements 1234 can have electrical communication therebetween through the tissue of the barrier, thus indicating contact with the barrier. In an exemplary embodiment, the specific impedance resulting from the tissue will be different than that which would be the case if the electrodes were contacting each other. Accordingly, in an exemplary embodiment, the hand tool 1220 is configured with a chip or some other logic circuit that can indicate whether or not the electrodes 1234 are in contact with the tissue of the barrier based on the impedance for example. In an exemplary embodiment, the hand tool includes a battery, such as a 1.5 V battery or a plurality of batteries to increase the voltage, or a 9 V battery, or can be provided with electricity from a 120 or 240 V alternating source that is common household electricity, and then the voltage can be stepped down (or up) and the current can be rectified if desired to provide a sufficient current and voltage source to enable contact determination. In an exemplary embodiment, an LED or the like is provided with the hand tool 1220 and can be in signal communication with the logic circuitry (and power source), and the LED will illuminate when the elements 1234 are in contact with the tissue. A sound source can be provided in addition to this or, alternatively, which sound source will indicate that the electrodes are in contact with the tissue. Note also that in some embodiments, the electrodes can be such that the electrodes are in contact with each other prior to contact with the tissue, and then contacting the tissue closes the electrodes to move away from each other, thus increasing the impedance between the electrodes. That can be the indication relied upon by the logic circuitry that the barrier has been contacted otherwise reached.

And note that in some exemplary embodiments, elements 1234 are not electrified or otherwise are not sensors per se. Instead, they can be simply flexible proboscis elements (akin to antenna on a bug) that have sufficient rigidity in combination with their flexibility to provide a tactile indication that there is some resistance to further movement of the termination through the middle ear cavity. In an exemplary embodiment, elements 1234 can be spring like components. And to further enhance the tactile experience, some embodiments can utilize a telescopic termination, where it is the termination that is moved relative to the handle 1130, and thus the overall mass that is being moved is lower, and thus the momentum associated with forward movement will be lower, thus providing greater tactile feedback once the elements 1234 contact the wall (more accurately, because of a lower mass, the contact of the elements 1234 with the wall will not be eclipsed by the greater mass of the overall tool). But then again, the resistance provided by elements 1234 can be such that the resistance could be significant in fact, and thus even with the overall mass of the tool, there is a statistically significant chance that the resistance that results from the elements 1234 vis-à-vis forward movement of the termination will provide sufficient indication to the user that the distal end of the termination 1150 is proximate the barrier between the middle ear and the inner ear.

In an exemplary embodiment, the diameter of the termination 1150 is set so as to have utilitarian value with respect to establishing a through bore in embodiments where the initial molding is also utilized as the ultimate custom ear canal guide. In an exemplary embodiment, the outer diameter of the termination 1150 can have a range from 0.5 mm to 6.0 mm, or any value or range of values therebetween in 0.05 mm increments, and an inner diameter of the termination 1150 can ever range from 0.1 mm to 5.0 mm or any value or range of values therebetween in 0.05 mm increments. In an exemplary embodiment, the inner diameter of the termination is utilized as the guide for a drill bit or the like or some other device that is utilized to extend through the custom ear canal guide, because, in some embodiments, the termination is cut at one or more locations corresponding to the ends of the custom ear canal guide, and the material that is flowed therethrough is drilled out or otherwise removed so that the interior of the portion of the termination it remains can be utilized as a guide for the tool that is extended to the cochlea during use.

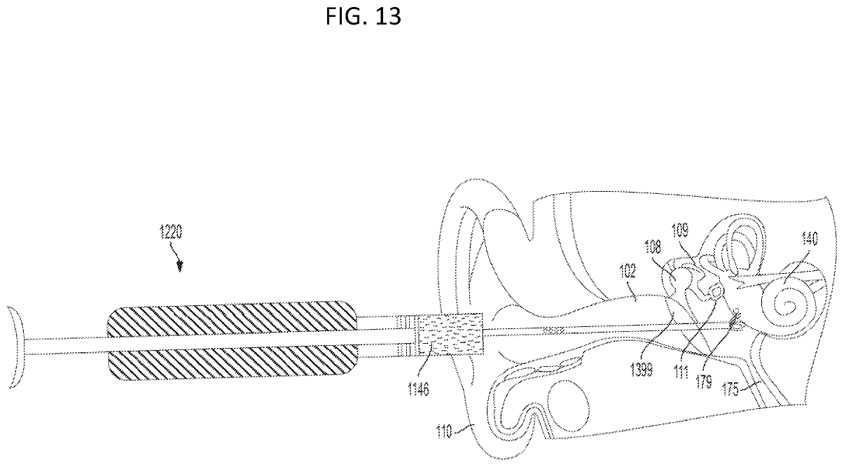

In use, first, access to the middle ear cavity 106 is established. This can be accomplished by utilizing a scalpel tool and cutting an incision through the tympanic membrane 104 (FIG. 13 depicts incision 1399). Typically, the incision will be between 0.25 mm (or less in some embodiments) and 3 or 4 mm or more or any value or range of values therebetween in 0.01 mm increments (e.g., 0.33 mm, 1.35 mm, 0.38 mm to 3.33 mm, etc.). That said, in some embodiments, as noted above, a grommet will be already located in the tympanic membrane. FIG. 13 presents the exemplary embodiment where the tool 1220 is located in part in the human (the specific human). Here, the termination 1150 extends through the ear canal 102 and through the tympanic membrane 104 and into the middle ear cavity 106, with the distal end of the termination 1150 proximate the target. In an exemplary embodiment, a borescope can be utilized to determine the location of the distal end of the termination 1150. Invasive and non-invasive imaging techniques or other invasive and non-invasive techniques can be utilized. Any device, system, and/or method that can be utilized to determine/estimate/approximate the location of the distal end of the termination 1150 relative to the target can be utilized in some embodiments.

And note that in some embodiments, the target is a result of the location of the distal end of the termination 1150. In this regard, in some embodiments, there can be wide ranges of locations of the target. In at least some exemplary embodiments, any location that will enable access from the middle ear into the inner ear without having deleterious effects or otherwise any location that has utilitarian value can be utilized. Accordingly, the results of the placement of the termination can be utilized to determine a target location. That is, the general target location will be known, but the specific target location may be adopted after implementing the techniques herein.

But in any event, FIG. 13 depicts the termination 1150 extending through the opening 1399 in the tympanic membrane 104, and the elements 1234 in contact with the barrier between the middle ear and the inner ear at a location at and below the round window niche. Again, the elements 1234 can be contacting at other locations. In the embodiments detailed herein, there is utilitarian value with respect to obtaining data indicating various features of the wall that establishes the barrier between the middle ear and the inner ear, and based on the determination of those features, identify the target. More on this below.

Having established in at least some exemplary embodiments that the distal end of the termination 1150 that is proximate the barrier (in some embodiments, a trial and error approach can be done—as detailed below, the teachings detailed herein can result in a negative of the surfaces of the barrier, and if the negative does not have the desired geographic features, the process can be repeated until the desired geographic features are obtained on the resulting negative), the molding material 1146 is pushed into the termination 1150 by applying pressure to the thumb support 1142 in a direction towards the handle 1130. This drives the plunger 1144 (or, in the double barrel embodiment, both respective plungers—or two separate handles can drive separate plungers) towards the termination (or towards the mixing chamber), thus driving the molding material, which can be unique specific ear impression material, into the termination. The termination 1150 is sized and the mentioned so that the ear impression material/molding material will exit the holes 1152 along the length of the termination, and thus flow into the outer ear canal 102. The overall design of the tool 1220 is configured so that the molding material (again, where a species can be a specific ear impression material) material will extend from the holes a determination to the wall surfaces of the ear canal 102. In an exemplary embodiment, the molding material will “fill” the outer ear canal 1102 with respect to a cross-section extending normal to the longitudinal axis of the outer ear canal and establish an earplug when cured. In some embodiments, that cross-section will not be filled per se, but a sufficient amount of the molding material will be located between the termination of the sidewall to implement the teachings detailed herein. And indeed, while the embodiment shown is presented in terms of a very diffuse or otherwise minimally controlled directionality of flow with respect to the molding material exiting the lateral sides of the termination, in some embodiments, conduits can be utilized to direct the molding material or otherwise channel the molding material towards the sidewalls. Indeed, in an exemplary embodiment, conduits can extend from the sides of the termination, which conduits can be sized and dimensioned to accommodate the standard human factors engineering sizes of an outer ear ear canal, where the molding material will flow out of the ends of those conduits and thus interface with the outer surface of the wall of the outer ear canal. Thus, the resulting molding material may be “globs” of material extending between the ends of the conduits and the surface of the wall, which globs may not contact each other with respect to a path that extends along the surface of the ear canal. And further, in some embodiments, while the termination is shown as having a uniform outer diameter (and, with respect to a determination having a standard wall thickness, thus a uniform inner diameter) from the cylinder 1199 to the termination, in an exemplary embodiment, the portion of the termination at the location of the holes 1152 can be a larger diameter portion of the termination, which places those holes closer to the surface of the ear canal relative to that which is the case with respect to the embodiments of FIGS. 11 and 12. Accordingly, the molding material “fills” a smaller area relative to that which is the case with respect to the embodiments of FIGS. 11 and 12. Providing that the overall trajectory of the termination can be located in a utilitarian manner prior to the transportation of the molding material so that the molding material contacts the outer surface of the ear canal, any arrangement that can have utilitarian value with respect to implementing the teachings detailed herein can be utilized.

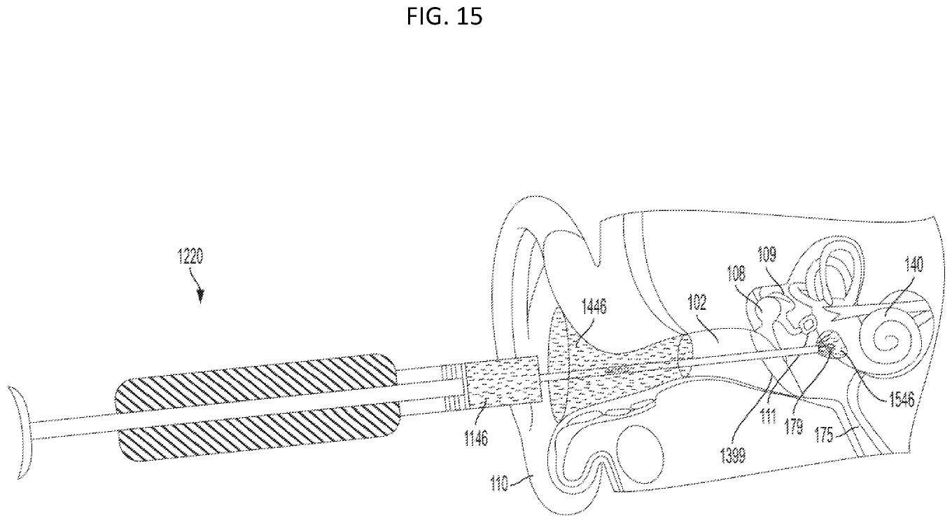

And with regard to “globs,” aforementioned “globs” can correspond to the “glob” that is located at the end of the termination, as seen in FIG. 15. (In an exemplary embodiment, the channels extending away from the termination can be many terminations themselves, and thus the effect at the end of the termination can be reproduced in an analogous matter along the length of the termination.) Here, there is a “glob” 1546 located at the distal end of the termination and “fills” the area between the distal end of the termination and the barrier between the middle ear and the inner ear at the location at the round window niche and the locations proximate thereof (in some embodiments, the boundaries of the “glob” 1546 do not reach the oval window and/or the ossicles, such as in embodiments where the ossicles are intact and function to provide at least a modicum of normal hearing, or at least where the ossicles are utilized with respect to other techniques, such as an implanted microphone in the cochlea) and/or otherwise located at least 0.5, 1, 1.5, 2, 2.5, or 3 millimeters or any value or range of values therebetween there from—in some embodiments, there is a shield or otherwise a barrier that prevents the therapeutic substance from reaching the oval window—and in some embodiments, the termination is sized and configured to direct the molding material away from the oval window—the end of the termination can be slightly curved or otherwise angled away from the oval window—and again, in some embodiments, a borescope can be utilized for otherwise be co-located with or even a part of the tool so that a visual image can be produced in real time to aid the user of the tool in preventing the flow of the molding material from reaching the oval window.

In any event, here, the molding material is transported to the wall of the ear canal and transported to the barrier between the middle ear and the inner ear. In an exemplary embodiment, there is utilitarian value with respect to the material being transported to the barrier coming into contact with some form of landmark thereof such as, for example, the round window niche or some other landmark that can be recognized from the resulting negative that results from utilizing the molding material (more on this below). In some embodiments, the landmark can in fact be the oval window and/or a portion of the ossicles if there is no utilitarian value with respect to that tissue vis-à-vis the specific human at issue or otherwise if the molding material, when cured, can be removed in a manner that does not have a deleterious effect on that tissue. And in some embodiments, the landmark can be an existing artificial component, such as a port that is located at a pre-existing cochleostomy (in embodiments where, for example, the goal is to access the port) or the extra cochlear portions of the cochlear implant electrode array for example, or a portion of a middle ear implant or some other device that is located in the middle ear. Any landmark that can have utilitarian value with respect to the teachings detailed herein can be utilized in at least some exemplary embodiments.

In any event, after a sufficient amount of the molding material comes in contact with the barrier and the surface of the wall of the ear canal, the molding material is permitted to cure or otherwise change from a first date to a second state, where the second state is a state where the molding material will have a memory of the surfaces with which the molding material came into contact with the molding material in the first state. By way of example only and not by way of limitation, the molding material can be a UV curing silicone. The molding material could be material used for RTV molding. The molding material could be wax or alginate, etc. Any molding material that can enable the teachings detailed herein can be utilized in at least some exemplary embodiments. In an exemplary embodiment, the application of heat can be utilized to cure or otherwise stiffen or otherwise harden the material. Thus, the material can be a material that hardens with the application of heat. In an exemplary embodiment, the time period of the transition from the first state to the second state (or the time period between the completion of transportation of the material to transformation to the second state can take less than or equal to 1, 2, 3, 4, 5, 6, 7, 8, 9, 10, 11, 12, 13, 14, 15, 16, 17, 18, 19, or 20 minutes or any value or range of values therebetween in 0.1 minute increments. In an exemplary embodiment, a general anesthesia and/or clamps can be utilized to hold the human still and/or hold the tools still during the curing period/hardening period or otherwise the transition period.

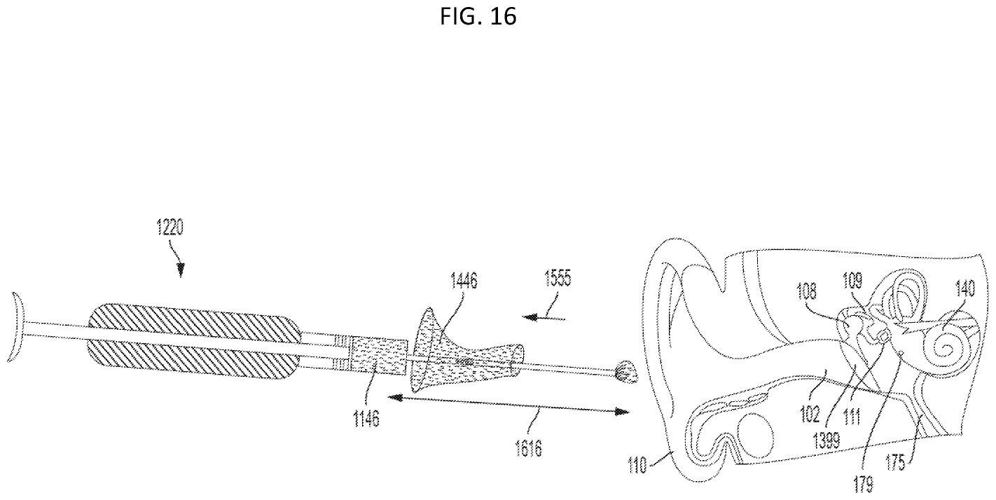

After the molding material, or at least a sufficient utilitarian amount, has cured or otherwise transformed to the second state from the first date (where the first date was the state of the material when the material was transported from the cylinder of tool 1222 determination and then into contact with tissue of the specific person), the tool 1120, or at least the termination 1150 (in an exemplary embodiment, the termination 1150 or the assembly of which the termination 1150 is a part can be configured to easily remove from the cylinder 1199, and in another embodiment, the termination can simply be broken off or otherwise cut—there could be frangible components or otherwise weakened components that will permit ease of breaking and/or ease of cutting—this can be done after sufficient amounts of the molding material have been transported into the outer ear and/or middle ear to enable the teachings detailed herein, where, for example, having the entire tool 1120 maintain in that position for the length of time that it takes for the material to cool would be less utilitarian than if the remainder of the tool, including the heavier and/or bulkier parts, could be removed and set aside while the material transforms from the first state to the second state) is removed by pulling such in the direction of arrow 1555 as seen in FIG. 16.

And in this exemplary scenario, the molding material that ejected from the tip of the termination 1150 “solidified” against the surface of the barrier between the middle ear and the inner ear. The solidification of the material is such that the material is soft and flexible enough to be pulled back out through the incision in the tympanic membrane without causing damage to the tissue thereof, or any other tissue for that matter. And in this regard, the material in the solidified state can be sufficiently soft and flexible so that if the material is contacting, for example, the ossicles, the material can be pulled away from the ossicles without damaging the ossicles. And note that in some embodiments, the material may not necessarily solidify, but the material will have a memory such that when the material is in a relaxed state, the memory results in a negative of the tissue where the material contact the barrier and/or a negative of the tissue where the material contacted the ear canal wall.

In an exemplary embodiment, a maximum diameter of portion 1546 of the material in the second state with respect to a plane parallel to a tangent surface of the barrier where the portion 1546 is in contact is about 1, 1.5, 2, 2.5, 3, 3.5, 4, 4.5, 5, 5.5, 6, 6.5, 7, 7.5, 8, 8.5, 9, 9.5, 10, 11, 12, 13, 14, or 15 mm, or any value or range of values therebetween in 0.1 mm increments. The maximum diameter could be larger. In an exemplary embodiment, the aforementioned maximum diameter is normal to the longitudinal axis of the termination. In an exemplary embodiment, the aforementioned diameter values exist with respect to diameters that are normal to each other and normal to the longitudinal axis of the termination and/or parallel to the aforementioned tangent surface.

In any event, FIG. 16 depicts an embryonic custom ear canal guide along with a custom barrier negative after the entire amount of the material in the second state is completely removed from the human. Element 1446 is a body having the termination extending therethrough, which outer surface of the body, or more specifically, which portions of the outer surface of the body that interfaces with the tissue of the human (here, the ear canal) have a negative imprint of the ear canal surface. And in the same vein, element 1546 has a negative imprint of the tissue that element 1546 contacted. That is, in this exemplary embodiment, element 1546 will be a negative copy of the barrier surface between the middle ear and the inner ear that can be reached from the tympanic membrane, such as, for example, the surface that can be reached in a straight line from the tympanic membrane (and a straight line through at least a portion of the ear canal—in an exemplary embodiment, the straight-line corresponds to a distance that is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mm or more (the latter figures are for a large ear canal), or any value or range of values therebetween in 1 mm increments along that line from the surface of the tympanic membrane facing the ear canal.

In at least this exemplary embodiment, because element 1546 extended over the round window niche 179, element 1546 will have a negative of the opening and/or the surface surrounding the opening of the round window niche.

In this exemplary embodiment, the distance 1616 between the surface of element 1546 that interfaced with the tissue of the barrier and the surface of element 1446 that faces away there from (or more accurately, the portion of the surface at the location of the termination/where the termination extends from the element 1446) will be an exact distance. And in this regard, the material that is used as a material that has dimensional stability, at least when cured. In an exemplary embodiment, the dimensional stability is stability that exist through a range of expected temperatures and/or humiditys. In an exemplary embodiment, the aforementioned exact distance 1616 will be maintained with respect to a value that is plus or minus 0.1, 0.2, 0.3, 0.4, 0.5, 0.6, 0.7, 0.8, 0.9, 1, 1.25, 1.5, 1.75, 2, 2.25, 2.5, 2.75, 3, 3.5, 4, 4.5, 5, 5.5, 6, 6.5, 7, 8, 9, or 10% plus or minus, or any value or range of values therebetween in 0.01% (and note that not all of these values necessarily correspond to dimensional stability—we are simply quantifying values here with respect to an embodiment).

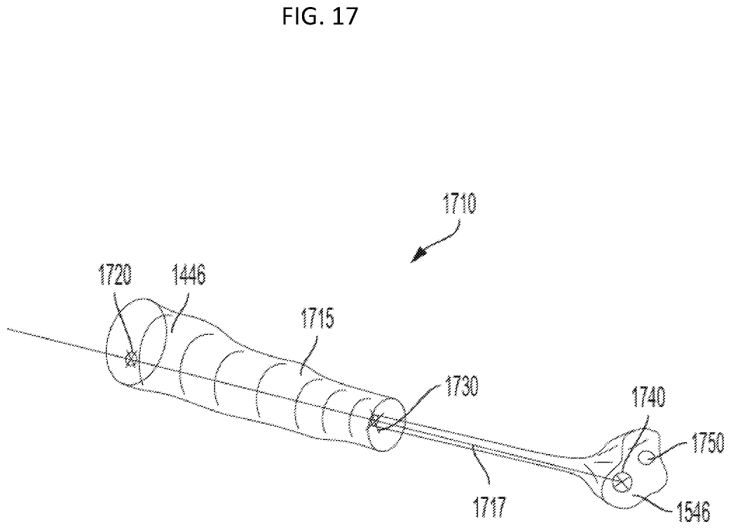

FIG. 17 presents an exemplary apparatus 1710 corresponding to either the resulting custom mold achieved from the removal of the material in the second state represented by FIG. 16, where the material removed is the material of apparatus 1710, or a separate apparatus that is made based on the material removed in FIG. 16. By way of example, the custom mold that was made can be either used directly, or can be scanned utilizing, for example, a 3-D analysis device (e.g., scanner using electromagnetic radiation (including visual) and/or a stylus-based device, etc.), and then reproduced based on the data set from the 3-D scanner, such reproduction can correspond to, for example, a 3-D printed apparatus. Alternatively, and/or in addition to this, a second mold can be made (e.g., using plaster, for example), which second mold corresponds to the actual contours of the tissue (it is not a negative) and utilizing the second mold, a second apparatus can be made. This latter aspect can have utilitarian value with respect to developing an apparatus that is made from a material that is more utilitarian than the material that was utilized to make the initial mold. Any device, system, and/or method that can enable the development of a custom ear canal guide that has utilitarian value according to the teachings detailed herein can be utilized in at least some exemplary embodiments.

In any event, apparatus 1710 will have a utilitarian feature, feature 1750, which feature is a landmark of the barrier between the middle ear and inner ear. In this exemplary embodiment, feature 1750 is the round window niche. From this feature the location 1740 can be deduced, which location corresponds to the target location on the barrier. And again, the target location is not necessarily a predefined location as much as it is a target of opportunity that is identified after the feature 1750 exists in apparatus 1710. And in this regard, based on the knowledge of prior study of the middle ear and inner ear, the person of ordinary skill in the art will know where a location that is utilitarian with respect to accessing the inner ear, such as via a drill, would be located relative to the landmark feature 1750, which feature again, could be the round window niche. Thus, based on the position of the landmark 1750 and/or the shape of landmark 1750 (the orientation/relative direction of the location 1740 can be deduced based on the shape of the landmark for example—by analogy, the location of Canada or Mexico, nay, the location of Toronto, Mexico City, Cancun or Acapulco, can be determined based on a map of the United States of America in isolation, providing that there is sufficient scale (hence the utilitarian nature of using the dimensionally stable material). In any event, based on the feature 1750 on portion 1546, which represents a negative of the barrier between the middle ear and the inner ear, location 1740 is determined one that surface representing the negative. Utilizing that as a point for axis 1717, another point is needed to fix the axis 1717. In here, this can be location 1720, which can be the center of the termination, or more accurately, the center of the through bore of the termination or the center of the outer diameter of the termination/central location thereof. Because the determination that was utilized was straight, the effect of the offset will likely be minimal in the greater scheme of things (offset relative to the location on the barrier between the middle ear and the inner ear where the axis of the termination extended through, which will be offset from the location 1740, or at least could be offset from the location 1740. That said, in an exemplary embodiment, location 1720 could be scaled relative to the offset from the longitudinal axis of the termination and the location 1740 chosen. In some embodiments, any axis passing through location 1740 and the proximal end of the body 1746 (which corresponds either directly or in the manner reproduced detailed above, with body 1446 FIG. 16), which has an outer surface 1715 that is a negative of the outer surface of the ear canal, that will result in a straight line that extends through the tympanic membrane at a location that is utilitarian and/or also extends through the entire ear canal without contacting tissue (other than the tympanic membrane to the extent that is considered part of the ear canal) when utilized in the specific human can be utilized in at least some exemplary embodiments. In this regard, it can be expected that because the termination 1150 that was utilized above passed through a portion of the tympanic membrane work was utilitarian to do so, reproducing a vector that will result in passage through that portion can also have utilitarian value. Accordingly, location 1720 can, in some embodiments, be determined relative to the longitudinal axis of the termination 1750, whether that be dead center at the location exiting the proximal end of body 1446, or offset therefrom.

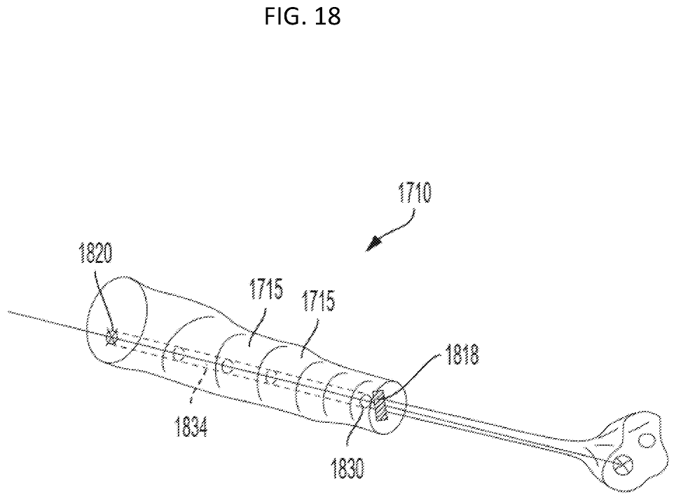

This then leads to the identification of location 1730, which should fall on axis 1717. Location 1730 in conjunction with location 1720 establishes the vector of the through bore that will be established extending through body 1446, and in this regard, FIG. 18 depicts a composite view showing cut 1818, where the portion of the apparatus 1710 that extends from body 1446 is cut off (in theory, it could be cut more distally), and then, based on locations 1720 and 1730, through bore 1834 is established that extends from the distal face to the proximal face of body 1446, where the through bore 1834 has a vector that would pass through the location 1740 if the element 1546 were still attached to body 1446 and oriented so that it is in the anatomically correct location.

The result is apparatus 1910, which has body 1446 having through bore 1834 and surface 1715, which surface is a negative of the surface of the ear canal. This is thus a custom ear canal guide that is custom to the ear canal of a specific human.

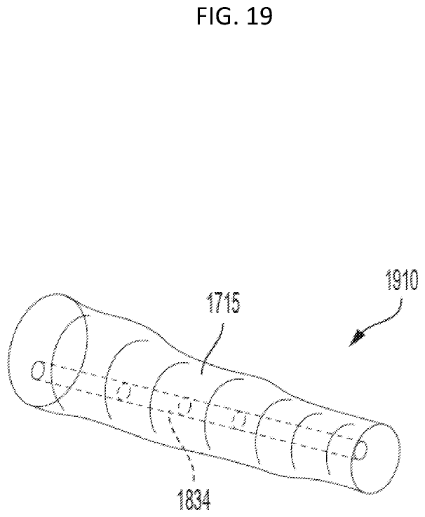

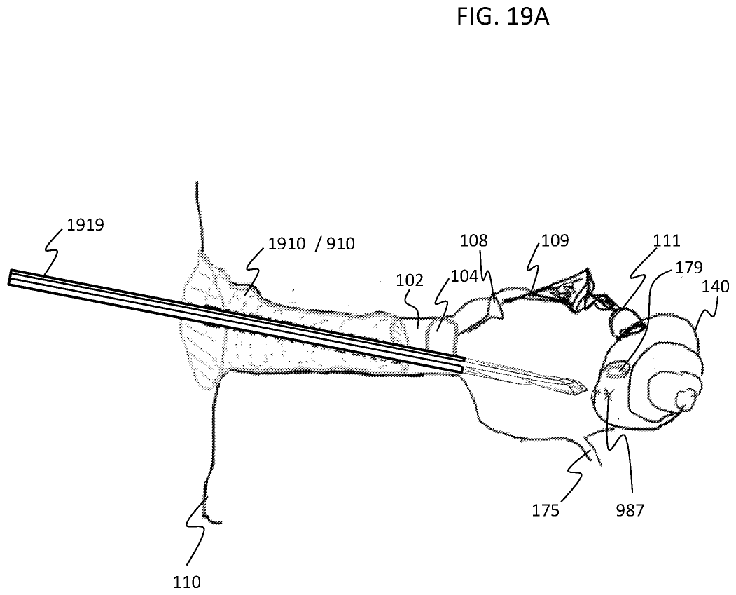

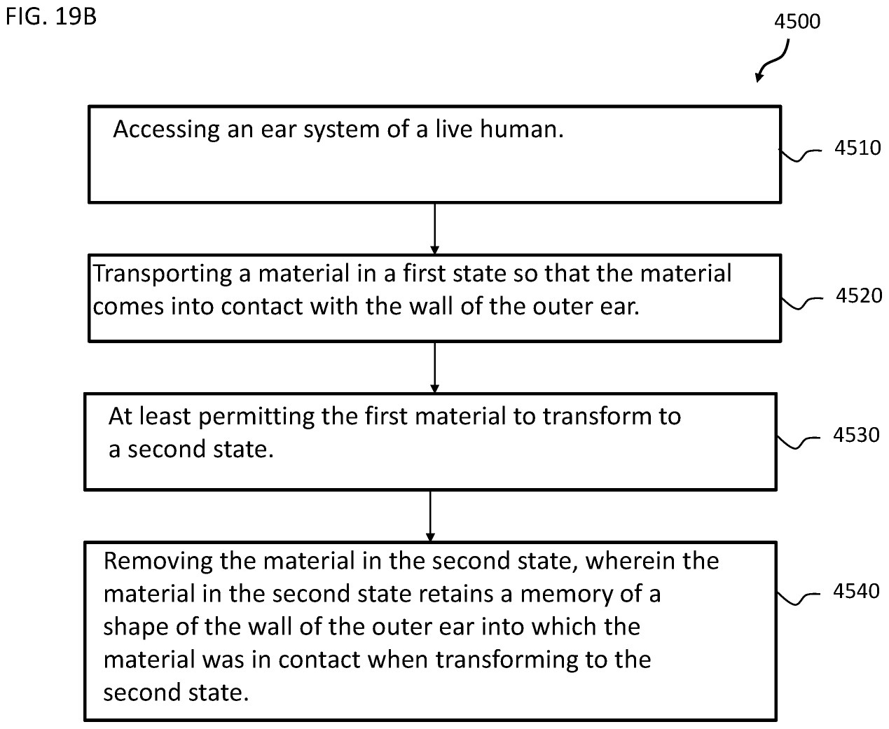

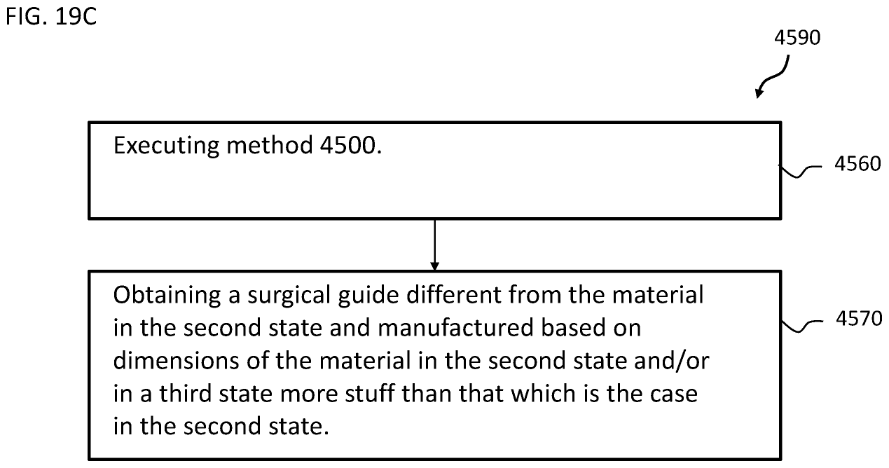

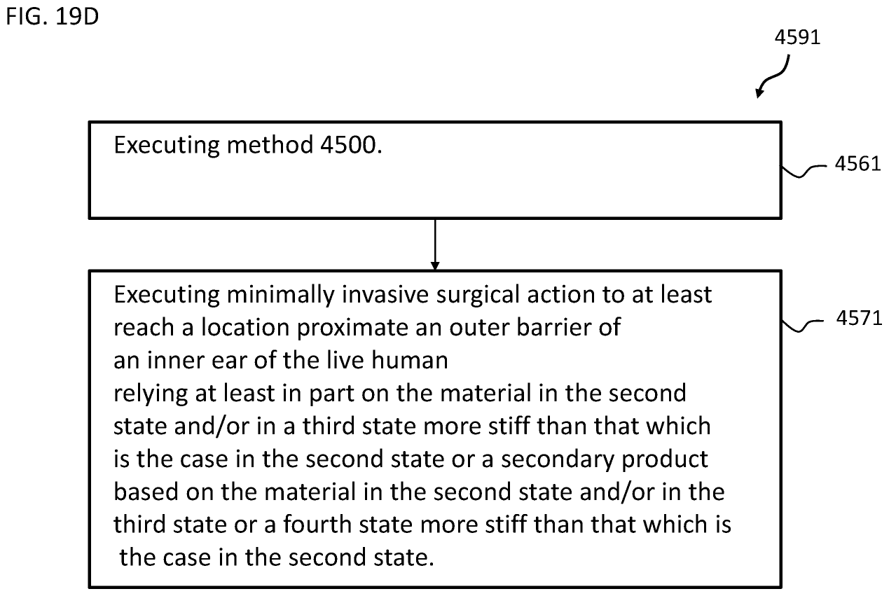

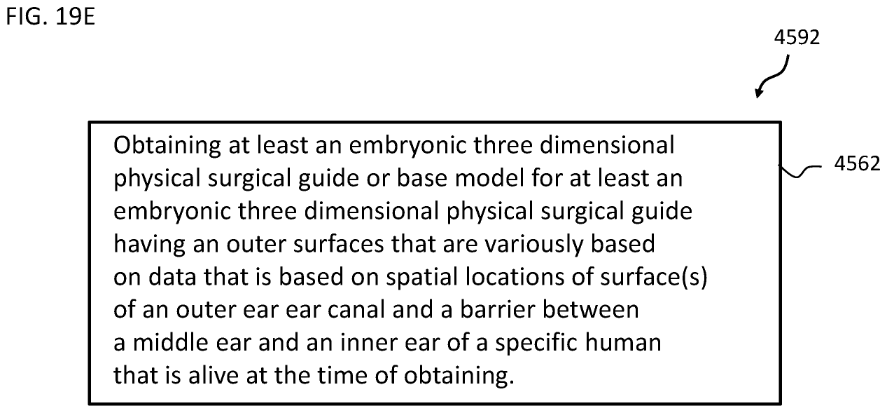

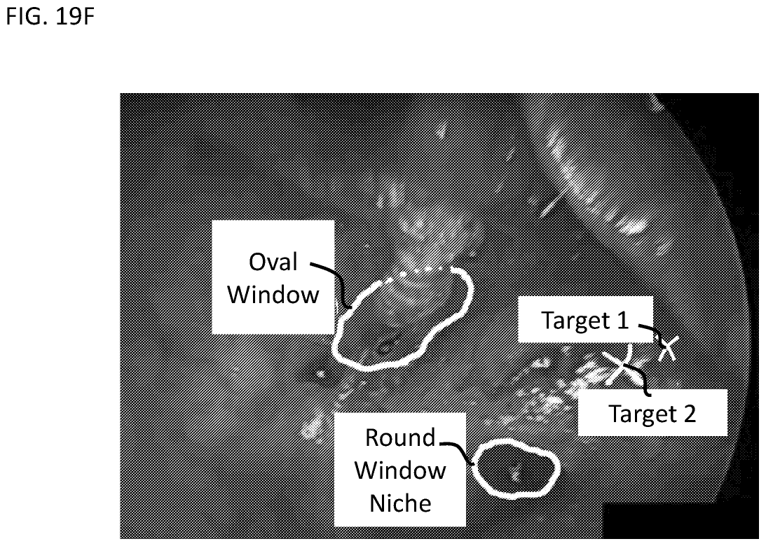

FIG. 19A depicts the utilization of custom ear canal guide 1910, which can correspond to custom ear canal guide 910 of FIG. 9 detailed above (and can correspond to use of the device that results from further refining body 1446 of FIG. 16 into a custom ear canal guide), to maintain the trajectory of a drill bit 1919. Here, the length of the through bore 1834 and the stiffness of the interior of the body that establishes the custom ear canal guide 1910 (which can be supplemented by, for example, a plastic and/or metallic tube or hollow cylinder—in an exemplary embodiment, the tube and/or hollow cylinder can be screwed into the bore that is established through the body—the bore that is established through the body can be oversized to accommodate what is arguably the equivalent of an elongate bushing) and the tolerances thereof relative to the outer diameter of the drill bit 1919 are such that the trajectory of the drill bit 1919 extends to the target location 987 when the drill bit 1919 is advanced through the through bore 1834. By attaching a drill motor or the like to the drill bit 1919, and rotating the drill bit 1919 accordingly, in applying sufficient forward pressure on to the barrier between the middle ear and the inner ear, a passage through the wall establishing the barrier into the inner ear, such as in duct of the cochlea can be established. This can thus correspond to establishing a cochleostomy in the cochlea.

And in this regard, embodiments can include a tube (which can be metal or plastic for example) that is slid over termination 1150 and can have holes aligned with orifices 1152 (or simply can have a large opening—the opening could be rectangular or the like and simply permit the passage of the material from the orifices to the ear canal—and indeed, it is noted that instead of orifices 1152, a large opening (or a plurality of such) could also be utilized for the termination). Any opening that will suffice that will still permit sufficient structural rigidity of the tool (termination and/or sheath/tube slid over the termination) can be utilized in at least some exemplary embodiments. In an exemplary embodiment, this tube or sheath will remain inside the through bore when termination 1150 is retracted/withdrawn after the impression material has solidified to form the inside wall of the through bore. In an exemplary embodiment, this can enable the establishment of a easy tool release from the impression material, provide a better defined through bore with exact inner diameter, an abrasion resistant inside surface and/or a defined (reduced) friction coefficient between a subsequently inserted surgical tool.

And with respect to trajectory, embodiments thus include a surgical guide having an internal bore to function as a stable and precise trajectory path for surgical instruments that are configured to enter through the ear canal and which eventually reach the middle or inner ear.

And in addition to the trajectory, the mold shown in FIG. 17 also defines the precise distance between point/location 1720 and the point/location 1740. In at least some exemplary embodiments, this precisely defined distance is utilized to adjust the surgical tool length such as a drill to define the drilling depth beyond point/location 1740 and/or to define over-insertion of a needle or catheter that is inserted into the cochleostomy created by the drill or into the passageway of a port into the cochlea. This can have utilitarian value in that it aids in avoiding unintentional damaging (or otherwise reduces the likelihood that such will occur in a statistically significant manner) of tissue of the human, such as, for example, the inner ear structures beyond the location 1740 such as the opposite wall of scala tympany or the basilar membrane.

And, in an exemplary embodiment, after establishing the cochleostomy at the target 987 utilizing the drill bit 1919, for example, the drill bit is removed and in some embodiments, the guide 1910 is removed and another tool is utilized to place a port that has a seal or otherwise can be sealed at the cochleostomy while in other embodiments a drug delivery device can be placed into fluid communication at the cochleostomy (and in some embodiments, the drug delivery device can be placed into fluid communication with the port). By way of example only and not by way of limitation, in an exemplary embodiment, the delivery tube 206 can be placed into fluid communication with the cochleostomy (or the port) so that the therapeutic substance can be delivered thereto instead of applying the therapeutic substance to the round window at the location outside the cochlea as is the case with respect to the embodiment of FIG. 5.

And while the above noted embodiment details removal of the guide 1910, in other embodiments, after the drill bit 1919 is removed, another tool is inserted through the passage of the guide 1910, and owing to the fact that the orientation of the through bore remains the same, the tool, if it is a straight tool, for example, will be directed to the cochleostomy, and, for example, if the tool is a port insertion tool, which port insertion tool is configured to carry a port, such as a threaded port, to the cochleostomy, and apply the torque to the threaded port so that port can be screwed into the cochleostomy, the guide can be utilized for the subsequent operation. And note also that the tool can be utilized to, for example, “ram” the tube 206 by way of example, into the cochleostomy, especially if the end of the tube 206 has a feature that will enable an interference fit, or when using a loose fit (relative to the tube and the female portions around the tube) where glue, such as surgical glue, is used to glue the tube in place using surgical glue and thus the establishment of some form of sealant the cochleostomy. (The glue only needs to hold the tube in place long enough for bone integration and tissue formation to secure the artificial component, at least in some embodiments.) And as will be detailed below, the guide 1910 can be utilized in conjunction with an endoscope or the like. And embodiments include methods of doing all of the above utilizing the custom ear canal guide.

And briefly, in this exemplary embodiment, the drill bit 1919 can be hollow so that irrigation fluid and/or cooling fluid can be transported through the drill bit to irrigate and/or cool the wall that establishes the barrier and/or cool the tip of the drill bit that is coming into contact with the barrier so as to not negatively affect the tissue owing to the friction forces resulting from the utilization of the drill bit.

Thus, in view of the above, it can be seen that embodiments can include a negative model of an inner wall surface of an ear canal of an outer ear unique to a specific person, which model has outer contours to the surface of the actual wall of the ear canal of the specific person. The outer contours can be the surface 1715 of the body of device 1910 above, for example. And note that in some embodiments, indicia can be provided on the surface facing outside the ear (the proximal surface) that would indicate the horizontal and/or vertical to provide the utility of giving a user a frame of reference when installing and otherwise utilizing the model. This is applicable to any of the custom ear canal guides detailed herein. Indeed, in an exemplary embodiment, the termination of the tool 1120 or 1220 has wings or structure that extends outward past the outer face/proximal face of the resulting material body 1446 in the second state that provide a reference for the horizontal and/or the vertical or whatever angle the termination was placed in (rotational), which angle can be measured and then transferred or otherwise utilized to establish the indicia on the face of the custom ear canal guide that the user sees when the user is inserting the guide into the ear canal.

In any event, consistent with the teachings detailed herein, the negative model includes a passage extending from a distal end to a proximal end of the model, the passage having a longitudinal axis that would bisect a barrier between the middle ear and the inner ear if the model was located in an anatomically correct manner in the outer ear. In an exemplary embodiment, this bisection occurs at the desired location 987 detailed above for example. In an exemplary embodiment, the bisection is less than, greater than and/or equal to 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2 3.3, 3.4, 3.5, 3.6, 3.7, 3.8, 3.9, 4, 4.5, 5, 5.5, 6, 6.5, 7, 7.5, 8, 9.5, 9, 9.5, 10, 10.5, 11, 11.5, 12, 12.5, 13, 13.5 or 14 mm or more (note that in many people the distance is typically less than 10 mm) or any value or range of values therebetween in 0.01 mm increments from the boundary of the round window niche as measured along the surface of the boundary between the middle ear and the inner ear.

In an exemplary embodiment, the distance between the end of the mold and the target 987 can be less than, equal to or greater than 30, 35, 40, 41, 42, 43, 44, 45, 46, 47, 48, 49, 50, 51, 52, 53, 54, 55, 56, 57, 58, 59, 60, 61, 62, 63, 64 or 65 mm or any value or range of values therebetween in 0.1 mm increments.

In an exemplary embodiment, the location 987 is a location that is on the other side of the barrier from the scala tympani. Other ducts can be accessed as well, upon changing the trajectory, in some embodiments.

In an exemplary embodiment, the custom ear canal guides detailed herein have utilitarian value with respect to maintaining a trajectory of a tool that has a straight longitudinal axis, such as the drill bit 1919. In an exemplary embodiment, the custom ear canal guides according to at least some exemplary embodiments are configured so that upon the application of 1 inch-pound (0.11298 Newton-Meters) of torque centered at a mid location of the through bore of the guide in at least one plane parallel to a longitudinal axis, where the torque is imparted onto a steel rod slip fitted into the bore that extends 1 cm out on either side of the custom ear canal guide, a maximum amount of rotation of the steel rod is less than or equal to 10, 9, 8, 7, 6, 5, 4, 3, 2, 1.5, 1, 0.9, 0.8, 0.7, 0.6, 0.5, 0.4, 0.3, 0.2 or 0.1 degrees or any value or range of values therebetween in 0.01° increments.

In an exemplary embodiment, the custom ear canal guides detailed herein also have utilitarian value with respect to maintaining a position in the XY coordinate system of a longitudinal axis of a tool that extends through the through bore of the guide. In an exemplary embodiment, the customer canal guides according to at least some exemplary embodiments are configured so that upon the application of one pound of force (4.4482216 Newtons) to the aforementioned steel rod at the center location, the maximum movement of the centerline of that rod in the direction of the applied force will be less than or equal to 3, 2, 1, 0.9, 0.8, 0.7, 0.6, 0.5, 0.4, 0.3, 0.2, or 0.1 mm, or any value or range of values therebetween in 0.01 mm increments, in at least one direction, and in some embodiments, such can be the case for all directions of application of force. In this regard, movement in the radial direction can be limited to the aforementioned values, and one or more or all directions normal to the longitudinal axis of the through bore.

And it is noted that while some embodiments herein are directed towards enabling movement of the tool along the longitudinal axis, it is noted that in at least some exemplary embodiments, such as where there is a cannula guide, the cannula guide can be fixed to the body of the custom ear canal guide, and thus some embodiments prevent movement in the longitudinal direction by any one or more of the aforementioned values when a force of 1 pound is applied in a direction parallel to and concentric with the longitudinal axis of such.

Accordingly, embodiments can provide fixation in three dimensions of a component that is extending through the body of the custom ear canal guide.

And note also that in some exemplary embodiments, the custom ear canal guides can be utilized as fixation jigs or the like, which can hold a tool and otherwise fix a tool in one or two or three dimensions.

In an exemplary embodiment, the negative model includes a passage extending from a distal end to a proximal end of the model, the passage having a longitudinal axis that would bisect a barrier between the middle ear and the inner ear if the model was located in an anatomically correct manner in the outer ear, the bisection being at a location below a round window and/or a round window niche of the person, the location below being relative to an orientation where an oval window of the person is above the round window. This does not mean that the location of bisection is perfectly aligned with the trajectory passing through the oval window in the round window, or even that it is below in a perfectly aligned head. This is simply to say, for example, that New Zealand is below China, even though if one heads directly north from Auckland, one will not hit China. And moreover, the same is the case with respect to looking at the Earth with the pole rotated 90 degrees from what is the “normal” view of the world—the below is relative to a frame of reference where the oval window is above. Indeed, say we look at the globe normally. Egypt would be above the United Kingdom based on a frame of reference where the United States is “above” the United Kingdom. And in this regard, FIG. 19F shows an annotated view of the barrier between the middle ear in the outer ear when looking from the middle ear, which shows a visually enhanced oval window and a visually enhanced round window niche, along with a target 1 and target 2. While almost all embodiments utilize a single target, it is noted that in some embodiments, there can be two or more targets. For the purpose of FIG. 19F is to show how the target one and target two would be beneath the round window niche where the frame of reference is the oval window being above the round window niche (here, because the oval window is to the left of the round window niche.

Another way of looking at this is that a line bisecting the center of the round window and the center of the oval window establishes a frame of reference where another line normal thereto that is at the portion of the border of the round window (or the round window niche in other embodiments where that is the reference) at the side of the round window opposite the oval window establishes a boundary where the location on the side of the boundary that does not encompass the round window is below the round window.

In an exemplary embodiment, the apparatus is located in the ear canal in an anatomically correct manner (meaning that the negative surfaces are proximate the proper surfaces), a termination or a drill bit or a guide cannula extends through the model and through a tympanic membrane of the person, such that a distal end of the termination or the drill bit or guide cannula is located at least proximate (which includes at or passing through in this context) a barrier between the middle ear and an inner ear of the person (e.g., the promontory of the cochlea). In an exemplary embodiment, the distal end of the termination is located within 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2 3.3, 3.4, 3.5, 3.6, 3.7, 3.8, 3.9, 4, 4.5, 5, 5.5, 6, 6.5, 7, 7.5, 8, 9.5, 9, 9.5, or 10 mm, or any value or range of values therebetween in 0.01 mm increments. In an exemplary embodiment, the termination is what extends through the model and the tympanic membrane, and in other embodiments, the drill bit extends to the model and the tympanic membrane, and in other embodiments, the guide cannula is what extends through the model.

In an exemplary embodiment, the negative model includes a passage extending from a distal end to a proximal end of the model, the passage having a diameter normal to a longitudinal axis of the passage that is greater than, less than and or equal to 0.5 mm to 8 mm or any value or range of values therebetween in 0.1 mm increments.

In an exemplary embodiment, the diameter is less than, greater than and/or equal to 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2 3.3, 3.4, 3.5, 3.6, 3.7, 3.8, 3.9, 4, 4.5, 5, 5.5, 6, 6.5, or 7 mm, or any value or range of values therebetween in 0.01 mm increments, this diameter being an open diameter (as opposed to another component being located therein, such as a bushing—this does not mean that there is no drill bit or termination therein—those are devices used with the apparatus, as opposed to part of the apparatus).

And in some embodiments, instead of the cannula guide, a catheter is utilized with the ear canal wall interfacing portion.

In an exemplary embodiment, the apparatus further includes a negative model of a portion of a barrier between a middle ear and an inner ear of the specific person. This can correspond to for example, the apparatus 1710 FIG. 17.

In view of the above, it can be seen that in an exemplary embodiment, there is a method including various method actions. FIG. 19B presents an exemplary algorithm for an exemplary method, method 4500, according to an exemplary embodiment. Method 4500 includes method action 4510, which includes accessing the ear system of a live human. This could be the ear canal of a human, and can include, in some embodiments, placing a slip or otherwise establishing an incision through the tympanic membrane of the human. Method 4500 includes method action 4520, which includes transporting a material in a first state so that the material comes into contact with the wall of the outer ear, such as, for example, the wall of the ear canal. In an exemplary embodiment, this can correspond to the above detailed mold material in the uncured state or otherwise the state that the material flows from tool that is going on in 1120 or 1220. (And as will be noted, in some embodiments, a two-part and/or a three-part or more arrangement can be used, where there are two or three different materials for example. As used herein, the transportation or otherwise the reference of a material can correspond to the material that is the one component of the two are three component material for example. And this material can change to a second state by interaction with a second or third material, even if the material is comingled with other material.)

And as can be seen from the above, in an exemplary embodiment, the action of transporting the material in the first state is executed also so that the material comes into contact with the tissue of a barrier between the middle ear and the inner ear. But then again, it is to be understood that in at least some exemplary embodiments, if the relative orientation of the termination relative to the location on the wall that is utilitarian with respect to whatever targeting action is executed is known or otherwise can be deduced, it may not be necessary to have the material contact the barrier per se. In this regard, the trajectory of the termination can be utilized to establish the trajectory of the through bore. In at least some exemplary embodiments a borescope or some optical system can be attached to the end of the termination of the tool 1120 or 1220. Moreover, the above-noted electrodes can be utilized somehow to sense the location of the distal end of the termination, such as, for example, voltage measurements between the distal tips of the electrodes and/or the impedances between such, etc. Also, in the example utilizing antennas or probe devices for example, the distal portions could yield a certain amount and the amount of yield would correspond to the contours of the barrier in contact therewith, and this yielding could be transposed into an electrical signal or some other signal that can be evaluated to determine the relative location of the distal end of the termination relative to a landmark, such as the round window niche.

But in any event, method 4500 includes method action 4530, which includes at least permitting the first material to transform to a second state. This second state can be the states detailed above, which can be a cured and/or a semi-cured state or partially cured state or a transition state. Any state that will enable the material to have memory of the interfacing tissues in a manner that will permit utilitarian value with respect to implementing the teachings detailed herein can be utilized in at least some exemplary embodiments.

Thus, method 4500 further includes method action 4540, which includes removing the material in the second state, wherein the material in the second state retains a memory of a shape of the wall of the outer ear into which the material was in contact when transforming to the second state. And of course, in at least some exemplary embodiments, the action of removing the material in the second state is such that the material in the second state retains a memory of a shape of the barrier between the middle ear and the inner ear of the live human into which the material was in contact when transforming to the second state.

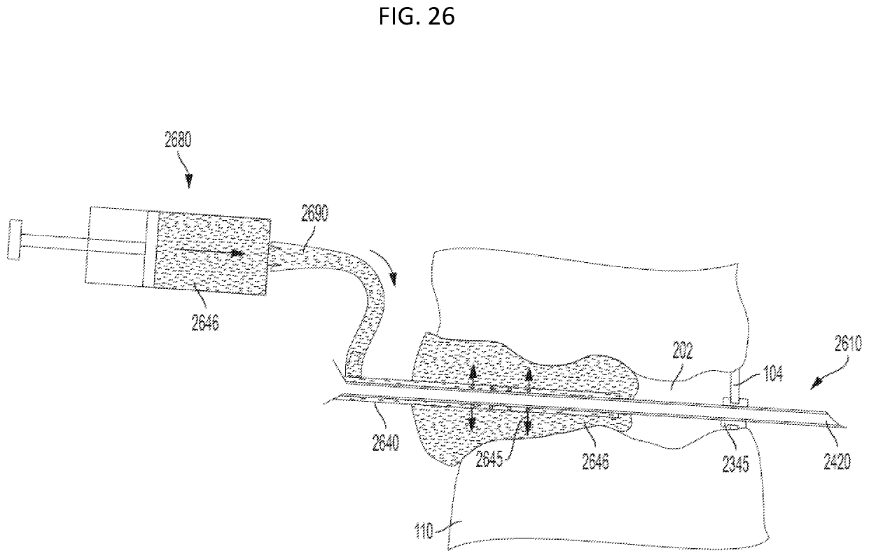

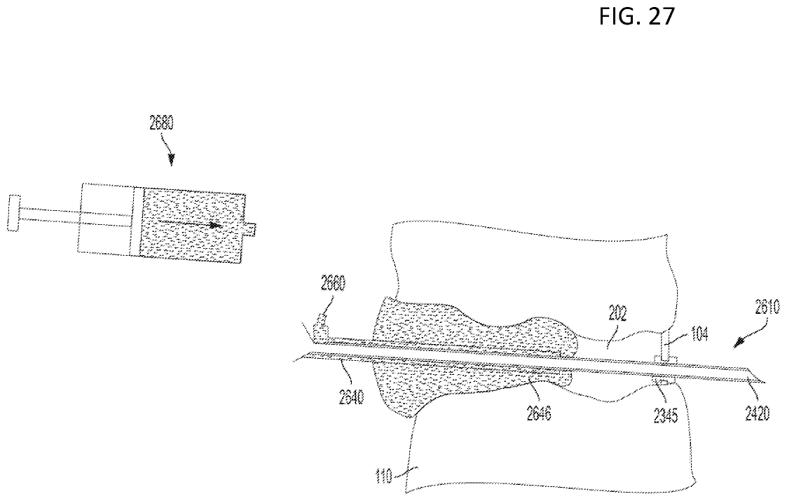

And jumping ahead a bit, it is noted that method 4500 is applicable to the arrangement of FIG. 26 for example (e.g., establishing device 2610), the details of which will be provided below.

Consistent with the above, in an exemplary embodiment, the material in the second state and/or in a third state more stiff than that which is the case in the second state (the material could be subject to further curing for example and/or could be subjected to an additional, different curing process by way of example) establishes at least a portion of an embryonic surgical guide configured for unique steady access to structure of an ear of the live human, such as, for example, the barrier between the middle ear in the inner ear of the human, and thus ultimately, when combined with a drill bit or the like, or when utilized with a channeling apparatus such as an elongate tube that can interface with or be coupled to a port or an existing passage through that barrier, a cochlea of the live human. This is a unique embryonic surgical guide and provides steady access that is unique because it is unique to that particular live human, as opposed to utilizing an embryonic surgical guide on a human other than the live human, which may for example provide the steady access to the structure, but would not provide the unique access. In an exemplary embodiment, this embryonic surgical guide is further refined (e.g., by establishing the through bore as detailed above for example) to establish the completed surgical guide detailed above.

Where the above exemplary method detailed in the prior paragraph establishes the material in the second state or in the third state as establishing the embryonic surgical guide, in an exemplary embodiment, there is a method, method 4590, as detailed in the flowchart in FIG. 19C, that includes method action 4560, that includes executing method 4500, and further includes method action 4570, that includes the action of obtaining a surgical guide that is a device that is separate from the material in the second state (e.g., the two devices could exist simultaneously) and manufactured based on dimensions of the material in the second state and/or in a third state more stiff than that which is the case in the second state. That is, in this embodiment, the material in the second state or in the third state is utilized in, for example, three-dimensional scanning or is utilized to make a mold that can be utilized to make the obtained surgical guide. This as opposed to the method actions in the preceding paragraph where the material in the second state or the third state is actually used as part of the surgical guide.

In an exemplary embodiment, the material in the second state and/or in the third state establishes a surgical template, and the material in the second state and/or in the third state is utilized as a surgical template.

And in some embodiments, there is an exemplary method, such as method 4591, as represented in the flowchart in FIG. 19D, which includes method action 4561, which includes executing method 4500. Method 4591 includes method action 4571, which includes, after executing method action 4540, the action of executing a minimally invasive surgical action to at least reach a location proximate an outer barrier of an inner ear of the live human relying at least in part on the material in the second state and/or in a third state more stiff than that which is the case in the second state or a secondary product based on the material in the second state and/or in the third state or a fourth state more stiff than that which is the case in the second state.

In an exemplary embodiment, the action of transporting the material in the first state includes transporting the material so that the material comes into contact with a barrier between the middle ear and the inner ear, and the material in the second state retains a memory of a shape of an outer surface of the barrier between the middle ear and the inner ear. And in an exemplary embodiment of this exemplary embodiment, the action of removing the material in the second state includes pulling a portion of the material that interfaced with the barrier between the middle ear and the inner ear through an incision in a tympanic membrane, a relaxed maximum diameter of the incision being smaller than a maximum diameter of the portion in a relaxed state that is compressed to fit through the incision. As detailed above, this can be because the material is compressible in the second state. But also, it is noted that in an alternate embodiment, a larger incision could be made that does not rely on compression, at least not as much compression, of the material. By way of example only and not by way limitation, the incision in the tympanic membrane could be large enough to retract the material from the middle ear without resistance. In an exemplary embodiment, the amount of resistance that is required to retract the material through the tympanic membrane is less than and/or equal to 20, 19, 18, 17, 16, 15, 14, 13, 12, 11, 10, 9, 8, 7, 6, 5, 4, 3, 2 or 1 Newtons or less or any value or range of values therebetween in 0.1 Newton increments. And in some embodiments, a reinforcing device (e.g., a tube with a circular or oval cross-section) can be utilized to fit around the incision so that the reaction forces provided against the tympanic membrane so as to press against the tympanic membrane as the cured material is being pulled therethrough so that the deflection of the tympanic membrane is limited.

In an exemplary embodiment, a catheter or a tube can be slid into the incision so that the force of compression is reacted against the interior of the tube (as opposed to directly against the incision). Indeed, in an exemplary embodiment, the catheter or tube is co-located and concentric with the termination, and the material flows through the termination and thus flows through the catheter tube to reach the barrier between the middle ear and the inner ear. A tool can be utilized to hold the catheter tube in place while the portion of the material in the second state is pulled through the tube so that the tube is not pooled outward out of the incision. Still, in an exemplary embodiment, the catheter tube can be slid forward at the time of withdrawal of the portion that interfaced with the barrier.

And it is to be understood that embodiments can include the utilization of the custom ear canal guide multiple times over days or weeks or months or years. In this regard, in an exemplary embodiment, there is a method that includes the action of, after removing the material in the second state from contact with the wall of the outer ear, executing a minimally invasive surgical action to at least reach a location proximate an outer barrier of an inner ear of the live human relying at least in part on the material in the second state and/or in a third state more stiff than that which is the case in the second state or a secondary product based on the material in the second state and/or in the third state or a fourth state more stiff than that which is the case in the second state. The method further includes, after executing the minimally invasive surgical action, executing a second minimally invasive surgical action to at least reach a location proximate an outer barrier of an inner ear of the live human relying at least in part on the material in the second state and/or in the third state or the secondary product based on the material in the second state and/or in the third state or the fourth state.

In this regard, it can be understood that the custom ear canal guide can be reused multiple times after the dimensions thereof are established. This can have utilitarian value with respect to executing one or more of the methods detailed herein without having to first check or otherwise verify that the tool utilized to reach the location proximate an outer barrier or to actually reach the barrier is on a proper trajectory. Thus, embodiments include executing one or more of the actions detailed herein without doing so at least in conjunction with one or more those actions (before and/or after and/or doing, depending on the embodiment).

It is noted that in at least some exemplary embodiments, execution of the minimally invasive surgical action can include passing a termination or guide tube of a tool through an existing grommet in tympanic membrane of the live human. This grommet could have been placed therein as part of the first minimally invasive surgical action, or before or after the first minimally invasive surgical action. In an exemplary embodiment, the minimally invasive surgical actions are executed repeatedly at a statistically significant rate that there can be utilitarian value with respect to adding a grommet repeated incisions in the tympanic membrane need not be made.

In an exemplary embodiment, a third and/or a fourth and/or fifth and/or a sixth and/or seventh and/or an eighth and/or a ninth and/or 10th and/or an nth minimally invasive surgical action according to any one or more of the above is executed utilizing the custom ear canal guide, where n equals any value or range of values between 1 and 10,000 in one increment. In this regard, such an embodiment can be utilized for the purposes of, for example, providing a therapeutic substance to the cochlea. This could be the case with respect to repeatedly accessing the port that is already in place, which port, when accessed, permits the transportation of therapeutic substance through the termination or a guide tube or whatever you are otherwise whatever transport device that is being utilized to the port and thus into the cochlea or otherwise into the inner ear.

It is noted that in at least some exemplary embodiments, the utilization of the custom ear canal guides can occur at various periods of time after the outer surface shapes and/or the trajectory of the through bores are established (this establishment may not be at the time of finalization—there could be more adjustments, but it is noted that the following temporal periods and qualifications can also be applicable from the time that the guide is finalized). By way of example, the first use and/or a second use and/or a third use and/or any of the nth uses detailed above of the guide can occur at least and/or equal to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 hours, or days, or weeks, or months, or any value or range of values therebetween in 1 increments after the establishment and/or finalization and/or after one or more of the precedent nth uses.

FIG. 19E presents a flowchart for another exemplary method, method 4592, which includes method action 4562, which includes the action of obtaining at least an embryonic three dimensional physical surgical guide or base model for at least an embryonic three dimensional physical surgical guide having surfaces that are variously based on data that is based on spatial locations of surface(s) of an outer ear ear canal and a barrier between a middle ear and an inner ear of a specific human that is alive at the time of obtaining. In an exemplary embodiment, the at least embryonic three dimensional physical surgical guide can correspond to apparatus 1710 of FIG. 17 or the resulting custom ear canal guide 1910 of FIG. 19. In an exemplary embodiment, the base model for at least an embryonic three dimensional physical surgical guide can correspond to the combination of the body 1446 and portion 1546 and attaching components of FIG. 16, by way of example. And moreover, the at least embryonic three dimensional physical surgical guide can correspond to the refined body 1446 which is actually used as the ultimate custom ear canal guide as detailed above.

In an exemplary embodiment, the outer surface can be based on the wall of the ear canal, and the surface that forms the bore for the drill bit, etc., can be based on the barrier, in accordance with the teachings above. With respect to the former, the outer surface is a negative of the wall of the ear canal, and hence the basis on the spatial locations of the surface of the outer ear ear canal. With respect to the latter, the trajectory of the bore through the guide is based on the spatial locations of the surface of the barrier, at least with respect to the guide for the embryonic guide that is produced based on a base model (e.g., the body 1446 and the portion 1546, etc.). With respect to a base model for at least an embryonic three dimensional physical surgical guide, again, the outer surface of the portions that relate to the outer ear ear canal are negatives of the surface of the outer ear ear canal, and thus based on the surface and the portions of the base model that are negatives of the surface of the barrier, and thus are also based on the surface.

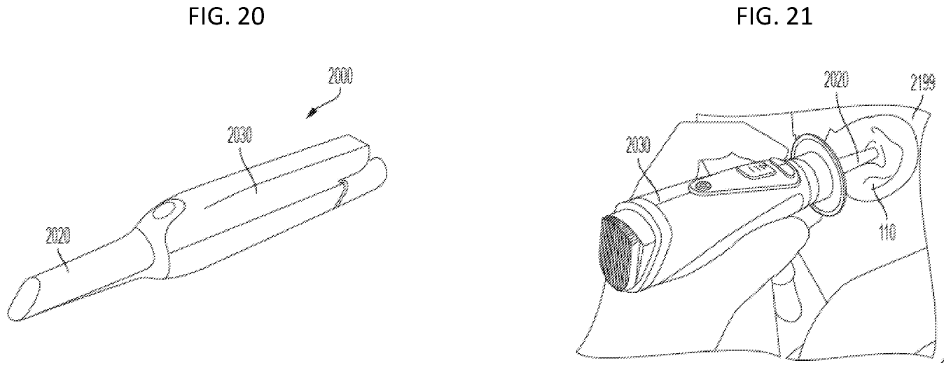

In an exemplary embodiment, the method can include actually developing the data, which can be developed by any of the techniques herein, such as, for example, utilizing the material 1446 in the second state after it is withdrawn from the ear canal. That said, in an alternate embodiment, scanning techniques can be utilized to model the ear canal and/or the barrier between the middle ear and the inner ear. In an exemplary embodiment, this can be established by a CT scanned or some other noninvasive skin. In an exemplary embodiment, this can be executed utilizing device 2010 of FIG. 20, which includes a handle 2030, and also includes a scan wand 2020, which is sized and the mentioned and otherwise configured to be inserted into an ear canal of a person 2199, as can be seen in FIG. 21. Device 2010 can be configured to develop an electronic data set of the interior of the ear canal in general, and the surface of the wall of the ear canal in particular. This can be done utilizing ultrasound and/or sonar-based and/or radar-based radiation, and thus the device 2010 can include one or more transducers and/or one or more transceivers that can output this radiation and/or receive this radiation, and based on the received radiation, developed a data set of the pertinent portions of the ear system, accordingly, such as, for example, the pertinent surfaces. In some embodiments of this device 2010, the transducers are configured to output radiation that can travel through the tympanic membrane or otherwise radiation of a wavelength to which the tympanic membrane is transparent, which radiation reflects from the barrier between the middle ear and the inner ear, and then travels again through the tympanic membrane to be received by device 2010, where it is utilized to construct the aforementioned data set. That said, consistent with the embodiments herein, there could be a passageway through the tympanic membrane, such as, for example, via an incision established for the initial purpose to extend a probe portion of the device 2010 through the incision, or via an opening in a grommet that is already located in the tympanic membrane and/or that has been put there for the initial purpose to extend the probe portion of the device 2010 therethrough. This probe portion could utilize some form of radiation that reflects off of the boundary between the middle ear and the inner ear in a manner concomitant with that detailed above. Alternatively, and/or in addition to this, the proportion could be tactile based and thus could be analogous to the utilization of a three-dimensional machine that utilizes a stylist to measure spatial locations of an object. And in this regard, the portion of the device that is located in the ear canal can also be tactile in nature. Any device, system, and/or method that can enable a virtual model of the ear canal and/or the barrier between the middle ear and the interior to be established can be utilized in at least some exemplary embodiments.

Accordingly, in an exemplary embodiment, the data is based on non-invasive electromagnetic scan(s) and/or tactile scan(s) of at least the ear canal. In an exemplary embodiment, the data is based on such scans of the barrier between the middle ear and the inner ear. In some embodiments, the data is based on both.

In view of the above, it can be seen that in some embodiments, the data is virtual data, while in other embodiments, the data could be physical model data.

The above said, embodiments of method 4562 include obtaining an already manufactured complete custom ear canal guide from a third-party that manufactured the guide (or the embryonic guide or base model, etc.), providing that the guide is a surgical guide (or model for such) and the guide (or model) has an outer surface that is based on data that is based on spatial locations of services of an outer ear ear canal and/or a middle ear of a specific human that is alive at the time of obtaining. In this regard, in an exemplary embodiment, the entity who executes the action of obtaining does not develop the data. Conversely, the entity who executes the action of obtaining can do so by obtaining data that was developed by another entity, and then manufacturing the at least an embryonic three dimensional physical surgical guide. By way of example only and not by way of limitation, a healthcare professional or the human himself or herself can utilize the device 2010, where the device can obtain data which can be utilized to create the data set where the data set can be the data obtained from the device, and then this can be transferred to an entity that has the ability to manufacture at least the embryonic three dimensional physical surgical guide. In an exemplary embodiment, this data set can be the three-dimensional data set that is utilized to 3-D print, and thus obtain, a physical surgical guide. This data set can be utilized to construct a mold that can be utilized to build the embryonic three-dimensional physical surgical guide.

In an exemplary embodiment, the action of obtaining includes obtaining the at least an embryonic three dimensional physical surgical guide and the data is based on a physical negative model of the outer surface of the wall of the outer ear canal (e.g., body 1446 and portion 1546 of FIG. 16).

In an exemplary embodiment, the action of obtaining includes obtaining a three dimensional physical surgical guide of the at least embryonic three dimensional physical surgical guide. Further, in this exemplary embodiment, for example, the surgical guide includes a passage extending from a distal end to a proximal end of the surgical guide, the passage having a longitudinal axis that would bisect the barrier between a middle ear and the inner ear of the specific human if the surgical guide was located in an anatomically correct manner in the outer ear, the bisection being at a location below a round window of the specific person, the location below being relative to an orientation where an oval window of the specific person is above the round window.

In an exemplary embodiment, the action of obtaining includes obtaining a three dimensional physical surgical guide of the at least embryonic three dimensional physical surgical guide, and method 4562 includes executing a minimally invasive surgical action to at least reach a location proximate an outer barrier of an inner ear of the live human using the physical surgical guide.

In an exemplary embodiment, an outer surface corresponding to one of the surfaces that are variously based on data negatively matches the outer surface of the wall of the outer ear canal of the specific human at at least a 50, 55, 60, 65, 70, 75, 80, 85, 86, 87, 88, 89, 90, 91, 92, 93, 94, 95, 96, 97, 98, 99, or 100% match rate, or any value or range of values therebetween in 0.1% increments even when not in contact with the wall of the outer ear canal of the specific human. In this regard, this differentiates from, for example, earplugs that are made of flexible material that conforms to the ear canal every time earplugs are placed into the ear canal, but which, when removed, deform away from that shape. And in this regard, it can be considered that in some embodiments, the guides (model or finished, depending on the scenario) have memory of the shape of the wall of the outer ear canal. And by “match rate,” it is meant that of the outer surface of the guide, at least an amount of surface corresponding to that percentage would contact the ear canal if placed therein without additional deformation (this discounts any deformation that may occur—this can be done with measurements of the ear canal and the guide in isolation).

In an exemplary embodiment, a finished guide has a durometer (e.g., Shore harness) of 20 to 80 Shore A or any value or range of values therebetween in 1 Shore A increment.

In an exemplary embodiment, the method includes obtaining the at least an embryonic three dimensional physical surgical guide (as opposed to the model—a module could be obtained as well) by making the at least an embryonic three dimensional physical surgical guide based on the data.

It is noted that any one or more of the teachings detailed herein regarding the method actions executed to obtain the surgical access guides/the custom ear canal guides according the teachings detailed herein can be executed as part of a surgical planning session or subsequent thereto before the actual surgery. In this regard, prior to the surgery, such as a surgery for implantation of a drug reservoir or a drug delivery device or a prosthesis, the surgery being minimally invasive or otherwise, the method actions detailed herein can be executed to prepare for that surgery. And indeed, in some respects, the methods executed to obtain the surgical access guides/custom ear canal guides are part of a minimally invasive surgical procedure, or at least part of a minimally invasive procedure, at least with respect to those that require passing a device through the tympanic membrane towards the target.

It is noted that in at least some exemplary embodiments, the utilization of the custom ear canal guides in a finalized state can occur at various periods of time after the outer surface shapes and/or the trajectory of the through bores are finalized, such as any of the time periods detailed herein (hours or days or weeks or months or years).

In view of the above, it can be seen that embodiments can include a three dimensional reconstruction of the patient's ear canal and middle ear space is either created directly through molding technology or indirectly through computer aided three dimensional scanning technology in combination with three dimensional printing (or some other method of manufacture). A channel can then be created through the three dimensional model with the entry on the outside facing surface of the three dimensional model and the exit on the towards the middle ear facing surface of the three dimensional model. Through identifying the target location on the barrier between the middle ear and the inner ear on the negative imprint of the surface of the barrier, the location of entry and exit of this channel is created so that when the three dimensional model (negative) is inserted back into the patient's ear canal it projects in a straight line onto the target location of the barrier. This channel then guides the insertion of surgical instruments like injection needles, drills and implants through the ear canal and middle ear towards the target location of the cochlea. Possible target locations could be the round window membrane for injecting drugs using a needle or an area on the promontory for drilling a cochleostomy to insert implants.

This surgical ear guide can be re-used/re-printed for subsequent access to the same location for re-administration of drugs or replacement of an implanted component. And thus, embodiments can provide a reusable surgical access guide, although it is noted that in other embodiments, the surgical access guide is not reusable or otherwise is not reused.

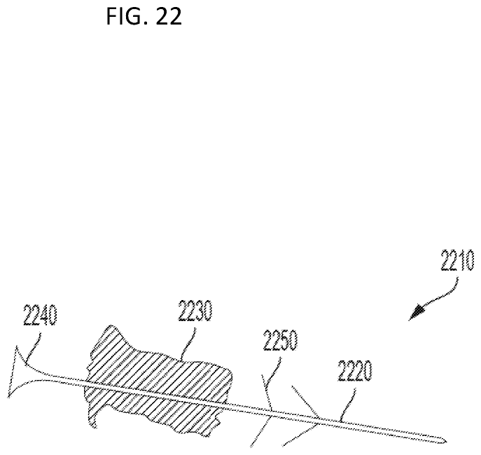

Embodiments such as those above provide a device that is configured to provide partially controlled access to the middle ear. The access is controlled in part owing to the limitations of the trajectory of the through bore. In other embodiments provide fully controlled access to the middle ear in that a component is part of the device that extends into the middle ear and limits the locations that a component that is inserted into the middle ear utilizing the device can reach. In this regard, FIG. 22 presents another exemplary device, device 2210, according to an exemplary embodiment. Here, device 2210 is a surgical access guide having a cannula configured for use in the ear canal. The cannula provides the fully controlled access just detailed, at least when the cannula is placed proximate the target, because any component inserted into the middle ear through the cannula and only “wonder” in the space between the tip of the cannula and the barrier between the middle ear and the inner ear or whatever tissue is the target. Device 2210 is a surgical access guide with a cannula that a user can insert into the ear canal and then fix the cannula 2220 into a position so that the trajectory of the guide cannula's middle axis is pointing towards a target location in the middle or inner ear (concomitant with the teachings above vis-à-vis using the through bore to align a component with a target—it is noted that embodiments of FIG. 22 and those below can include any of the teachings detailed above with applicability to the teachings of the embodiments of FIG. 22 and beyond, and vice versa unless otherwise noted, providing that the art enables such—for example, a feature disclosed for an embodiment of FIG. 19 is applicable to that of FIG. 22, and vis-a-versa). Fixating the cannula 2220 can be achieved by filling the space between the cannula and the ear canal with a molding foam/paste such as ear impression material, for example, that sets/cures. The fixed cannula guide 2220 then provides access to for surgical tools to be introduced through the ear canal in a desired trajectory, and in some embodiments, repeatedly (after the entire component is removed and/or while the component remains in the ear canal). Some embodiments of the device of FIG. 22 (and the other devices herein) enable single or repeated, minimally invasive, drug delivery procedures, endoscope insertion, and/or drill bit insertion, to the middle and/or inner ear.

More particularly, the teachings herein enable a surgical access guide cannula where a user can insert the cannula into the ear canal and then fix the cannula into a position so that the trajectory of the guide cannula's middle axis is pointing towards a target location in the middle or inner ear. The fixed guide cannula, as noted above, provides access to for surgical tools to be introduced through the ear canal in a desired trajectory to, for example, pierce the round window membrane and/or create a cochleostomy at the cochlea's promontory to access scala tympani. In some embodiments, because the cannula is fixed in place, surgical tools can be introduced multiple times targeting the exact same location. This can be utilitarian for using a first tool to create a cochleostomy, then, during the same procedure, inserting a second tool to introduce a fixture or port which can be fitted with a drug releasing implant, and embodiments include methods of doing so. The same guide cannula can be used to refill a reservoir of a drug releasing implant.

In an exemplary embodiment, initially, a grommet is placed in the tympanic membrane, such as, for example, in a quadrant away from the ossicular chain to avoid structural damage thereto. The grommets can be those that are used to ventilate the middle ear space in chronic otitis media patients, for example. Other, specific use grommets can be utilized as well. And the use of a grommet is optional. Instead, a simple incision can be used, which incision will heal or will be closed at the end of the procedure.

With respect to FIG. 22, for example, it can be seen that there is a guide cannula 2220 to which is attached support structure 2250, which can be in the form of wire antenna like components that will generally support the cannula 2220 when the canola is initially inserted into the ear canal and inserted through the tympanic membrane. This can prevent or otherwise reduce downward pressure on the incision of the tympanic membrane and/or on the grommet therethrough, and otherwise can prevent or otherwise reduce the likelihood that the portion extending into the middle ear will contact the ossicles or other structure and the middle ear that is undesired to be contacted, such as, for example, the oval window and/or the round window in the case of a scenario where the tissue that is the target is located elsewhere, such as below the round window in accordance with the teachings above.

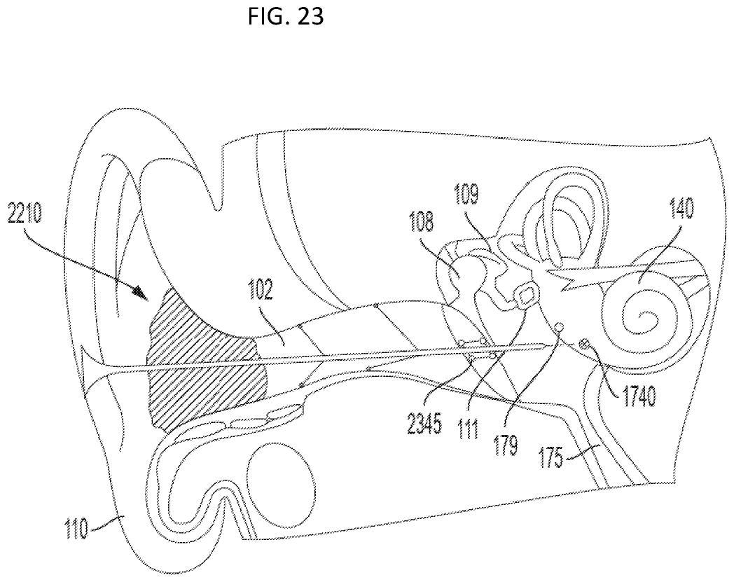

Support structure 2250 could be in the form of flexible plastic and/or metallic cones and/or segmented cone portions (e.g., a cone can be divided into three or four or five equal parts about the 360 degrees, thus permitting the outer circumference of the former cone to collapse or otherwise moved towards the cannula 2220 to facilitate insertion into the ear canal and/or removal from the ear canal. Any type of support structure that can have utilitarian value can be utilized. After this, the molded support structure 2230 is molded in place in the ear canal around the cannula 2220 to otherwise fix/maintain the trajectory of the canola and/or to hold the cannula in the X and Y direction (while permitting in some embodiments the cannula 2220 to move in the axial direction). More on how the support structure is molded below, but first, FIG. 23 presents an exemplary embodiment where the guide device 2210 is located in the ear system of a human and aligned with a target 1740 on the barrier between the middle ear and the inner ear, as represented by the longitudinal axis of the cannula 2220 extending to the target 1740. And in this embodiment, the exemplary grommet 2345 is shown in the tympanic membrane, with the cannula 2220 extending through the grommet.

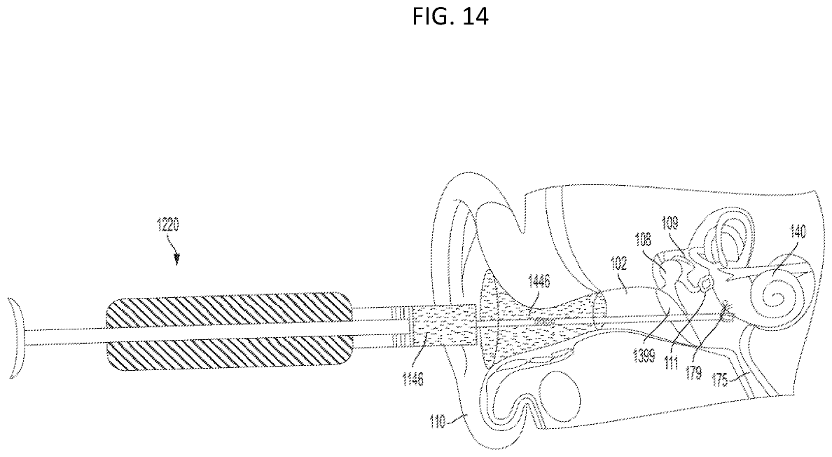

And it is noted that in at least some exemplary embodiments, the support structure 2250 may not be used at all in the first instance, and otherwise may not be present, as is depicted by way of example in the embodiments of FIGS. 26 and 27. This is consistent with any feature disclosed herein that can be utilized with or otherwise explicitly not utilized with any other feature disclosed herein unless otherwise noted providing that the art enables such. And with regard to the embodiments of FIGS. 26 and 27, here, we see cannula (which includes a termination) 2420 of an exemplary guide 2610, where the termination of the cannula includes the sharp chamfered edge, while it is noted that in other embodiments, the termination has a blunt end, extending through a grommet 2345, where the cannula includes an outer shroud 2640 that establishes a hollow space between the outer wall of the channeling tube (the part that would be present without the shroud) of the cannula 2440 which permits mold material to flow therein and down the longitudinal axis of the cannula 2440. More particularly, the cannula 2420 includes an attachment port 2660 that enables connection with a mini hose/flexible tube 2690 from a syringe 2680. The attachment port 2660 is in fluid communication with the hollow space established by the shroud 2640 noted above. When the plunger of the syringe 2680 is pushed forward, the mold material 2646 flows out of the cylinder of the syringe 2680 to the attachment port 2660, and then into the hollow space established by shroud 2640. The mold material 2646 then flows, under pressure, down the length of the channeling tube 2440 of the cannula, and then flows out orifices in the shroud, represented by arrows 2645. The mold material 2646 then “fills” the space between the cannula 2420 in a manner concomitant with the mold material detailed above with respect to the embodiment of FIGS. 13 and 14. The result is seen in FIG. 26, with a support structure 2646 established by the mold material, which mold material can be in a different state (e.g., cured) relative to that which was the case when the material flowed from the shroud. Again, UV curing can be utilized by way of example. In an exemplary embodiment, a temperature difference can be utilized to change the state, such as, for example, inserting a device in the conduit of the cannula that is at a higher temperature or lower temperature than the mold material, but which temperature will not deleteriously effect the tissue of the live human. Then, the syringe 2680 along with hose 2690 can be detached from the cannula as seen in FIG. 27, and thus the cannula is now a guide cannula in the live human ready for use to reach the target in the middle ear.

And it is noted that in an exemplary embodiment where the target is identified or otherwise located, the methods herein can include the action of relying on visual feedback based at least in part on an artificial device (e.g., an endoscope or a laser pointer) to identify a trajectory through the material in the first state and/or second state, which trajectory aligns with the target, such as the target on the barrier between a middle ear and an inner ear of the human. And as detailed herein, and endoscope or the like can extend through the material that is located in the ear canal while the material is changing from the first state to the second state so that the alignment/trajectory through the material can be established. By way of example only and not by way limitation, in an exemplary embodiment, a tube, such as a plastic tube, can be located adjacent the termination 1150 in the ear canal, and an endoscope or the like can pass through this tube, and can also extend through the tympanic membrane towards the target. The material can then flow into the termination 1150 and thus out of the orifices thereof, and into the ear canal, which material can surround this plastic tube, albeit offset from the termination. The tube protects the endoscope from contact with the material in the first state, and enables the endoscope to be easily withdrawn through the material in the second state. The tube would thus remain in the material in the second state, and could be reused for endoscopic purposes or otherwise to pass a laser pointer or the like therethrough to illuminate the target during subsequent uses. That said, the tube could be abandoned or otherwise not use subsequently because the trajectory would be established. Moreover, while the embodiment above utilized a tube to protect the endoscope, or otherwise maintain the material from the endoscope, in an alternate embodiment, a tube may not be used, and the material can come into direct contact with the endoscope. In an exemplary embodiment, the endoscope could be coated with a material or otherwise the material that transitions from the first state into the second state is a material where, in the second state, the endoscope is easily remove there from her otherwise can be removed there from without damaging the trajectory or otherwise the main functionality of the device.

Moreover, in an exemplary embodiment, the portion of the endoscope that extends through the material could be sacrificed in a manner analogous to that detailed herein with respect to sacrificing the portion of the termination (it could be cut off for example). Any device, system and/or method that can enable the utilization of an endoscope can be utilized in at least some exemplary embodiments, and, in some embodiments, the endoscope could be snaked through the termination, and otherwise removes with the removal of the termination.

Thus, in an exemplary method, it can be seen that a user inserts the guide cannula without the fixation material into the outer ear canal, blindly, or with a device that permits the user to see what is in front of the guide cannula's distal opening. This can be done using an endoscope that has a screen, or by directly looking through an eyepiece mounted onto the proximal end of the cannula, for example—to get a live view on a screen, the endoscope, along with a light source can be inserted into the cannula. Other tools such as irrigation tools to remove any obstructions that might be in the way of the target location, etc., can also be contained in the cannula or used with the cannula. In this exemplary embodiment, the guide cannula is advanced so as to, for example, pierce the tympanic membrane with its sharp tip (if present), or a surgical tool is advanced through the cannula to create a myringotomy, or in some embodiments, the termination of the guide cannula is extended through a pre-existing fenestration, grommet or some other entry port in the tympanic membrane. Any device, system, and/or method of accessing the middle ear through the tympanic membrane can be utilized in at least some exemplary embodiments.

In this exemplary method, the guide cannula is then positioned at a desired trajectory (direction) and/or distance to a target location (in some embodiments, because the arrangement permits the axial movement of the cannula while fixing the radial movement of the cannula, the distance may be generalized or may not be a concern at all initially) on the tissue in the middle or inner ear by aligning the center point in the field of vision onto the target tissue location. To align the center point with target tissue, a crosshair marking the center can be shown on the screen/field of vision when using an endoscope, by way of example.

In an exemplary embodiment, there can be a laser that is utilized to mark the tissue that is in a straight trajectory parallel to the guide cannula's middle axis in front of the cannula, by way of example only and not by way of limitation.

Once the guide cannula 2420 is in the desired location pointing towards the tissue target location, the user moves (e.g., by injection) a molding material, such as an ear impression material, such as a foam or paste, through a dedicated channel with exit holes in a position to fill the space between cannula and the inner surface of the ear canal at the entry of the ear canal and approximately 10 mm in either direction. (And it is noted that the channels detailed above can also be used in some embodiments—instead of the orifices, the shroud has pipes that channel the material towards the wall of the ear canal—again any feature disclosed herein can be combined with any other feature disclosed herein providing that the art enables such unless otherwise noted.) And it is noted that while the molding material (again, which can be ear impression material can be applied through channels and openings in the guide cannula as detailed above, in some other embodiments, a separate injector is utilized. That is, a standard guide cannula can be used, and the syringe 2680 and accompanying outlet device (e.g., hose, or termination) is utilized that is never connected to the standard guide cannula. The molding material in then solidifies (either by additional action or by normal temporal effects) and secures the guide cannula in a fixed position relative to the ear canal (at least in the radial direction and/or vis-à-vis trajectory). This can be utilitarian in that if the human's head moves, the trajectory of the guide cannula remains the same or at least effectively the same.

In an exemplary embodiment, the guide channel is used to insert endoscopes, irrigation, laser, drills, burrs, needles, fluidic cannulas and/or other surgical tools as well as implants such as drug containing pellets, polymers, nanoparticles, microspheres, drug solution and suspensions, gels, film forming agents etc., and embodiments include methods that include insertion of one or more of these things using the cannula.

And, for example, after completion of the procedure, the guide cannula and the solidified “plug” (or material in the second state) can be pulled out in one piece. In some embodiments, a solution can be applied to the solidified/cured plug to liquefy and/or dissolve or otherwise soften the material to make removal easier.

Some embodiments include a procedure that is to be performed repeatedly, such as, for example, with some sort of port or some sort of refillable drug reservoir which has been implanted. In such embodiments, in some instances, the molded plug might be made from a higher quality material to be reusable and/or it is scanned in using a 3D scanner to have its geometry as digital file for repeated 3D printing or injection molding. And in this regard, irrespective of the embodiment, it is noted that the component that interfaces with the ear canal wall can be repeatedly manufactured utilizing the underlying data that is obtained regarding the surface structure of the ear canal wall and/or the data relating to the barrier between the middle ear and the inner ear. And thus, embodiments can include executing the minimally invasive surgical procedures detailed herein in accordance with the numbers detailed herein where the body that interfaces with the tissue of the middle ear is created a new for at least one or all of those surgical procedures.

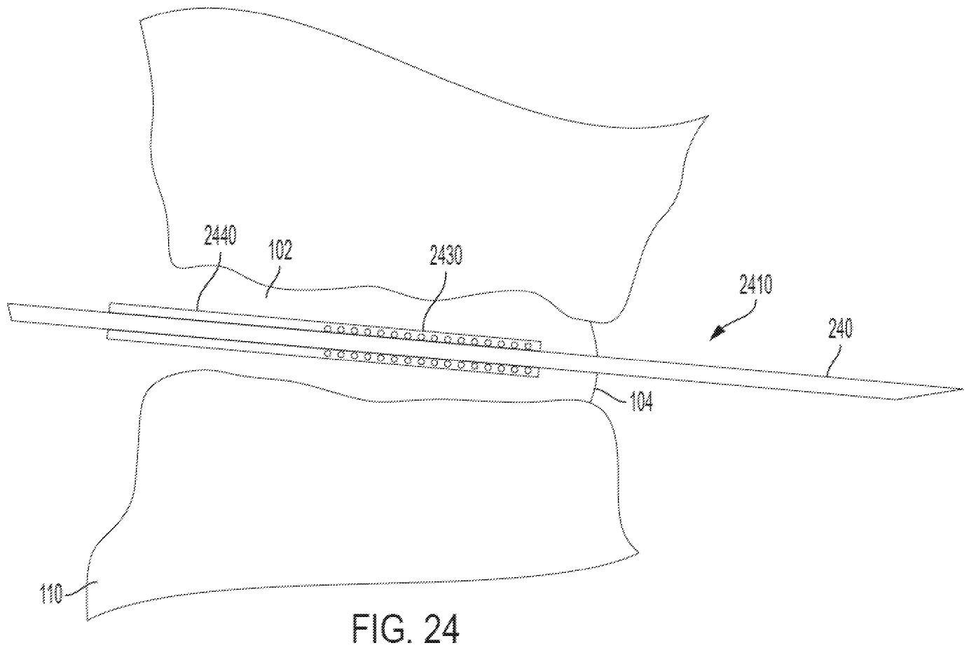

Also, as an optional embodiment, the guide cannula 2220 can be inserted into the ear canal and anchored against the inner surface of the ear canal using a mechanical mechanism such as a stent like structure. This mechanical mechanism supports the weight of the cannula and can avoid or otherwise reduce the likelihood of damage to the tympanic membrane. This can allow the guide cannula to be moved in radial, backward and/or forward direction and can be in some embodiments, only limited by the space available in the ear canal before it is fixed in a desired position in a subsequent action. And in this regard, FIG. 24 depicts an exemplary device 2410 that includes a cannula 2420 (the termination is shown) extending through the tympanic membrane 104. Here, a movable shroud 2440 encloses a compressed stent 2430 located in the ear canal 102. Device 2410 is configured so that after the cannula 2420 is position about where the user wants the cannula to be, the shroud 2440 (which can be a hollow tube or cylinder) is withdrawn/pulled back in the direction of arrow 2599, permitting the stent 24302 expand to a relaxed (or more relaxed) state as shown. Portions of the stent 2430 can be attached to the surface of the cannula 2420 so that the stent supports the cannula at the desired trajectory and/or location (some embodiments permit axial movement, and some limit such/forbid such) and otherwise fixes the cannula in such a state.

In view of the above, it can be seen that in an exemplary embodiment, there is an apparatus, comprising, a hollow medical component (e.g. the cannulas/terminations, or other hollow components used to reach the target location), and a support structure (e.g., any of the molded material component/stents detailed herein), distinct from the hollow medical component and supporting the hollow medical component, the support structure being configured to interface with an interior of a unique ear canal of a specific human and support the hollow medical component in a fixed trajectory relative to a target in a middle ear and the specific human, which target can be, a target on the barrier between the middle ear and the inner ear as noted above, which can be the promontory of the cochlea for example.

In an exemplary embodiment, the hollow medical component is a surgical guide (such as a cannula) or a termination (and the two are not mutually exclusive). In an exemplary embodiment, the hollow medical component extends at least and/or equal to 30, 40, 50, 60, 70, 80, 90, 100, 125, 150, 175, or 200 mm or more, or any value or range of values therebetween in 1 mm increments. An interior diameter of the hollow medical component (the diameter of the hollow portion) can be between 0.5 mm and 8 mm or any value or range of values therebetween in 0.1 mm increments.

In an exemplary embodiment, there is a variant of device 1910 that includes a plurality of lumens/passages. For example, this can have utilitarian value with respect to having a passage for an endoscope for example which can be utilized to provide a live view of the barrier between the middle ear and the inner ear, and then having a passage for example, for the surgical tool, such as the drill, the catheter, the port applicator, etc. In at least some exemplary embodiments, the one passage can be the “accurate” or otherwise the precision passage, which would be utilized for the working tool, and thus could be referred to as the “working passage,” and the other passage could be a less precise passage, or otherwise created in an offset or parallel manner (or an angled/acute angle manner knowing where the ultimate location will be) because the practitioner would understand the general if not the specific area where the endoscope or otherwise the tool that does not require the precision associated with the tool utilized in the working channel would be located or otherwise would need to be positioned to have utilitarian value.

Indeed, there could be a third channel and/or a fourth channel (passage) utilitarian value for such could be a channel for irrigation and/or a channel for suction. And indeed, there could be to “working channels,” but where only one is needed for the high precision, such as that associated with the drill, and a less precise passage is utilized for the catheter for example. Moreover, it could be that the bore diameters of the passages, or more specifically, the working passages, could be such that two different diameters are needed. For example, if a smaller diameter for the more precise drilling is required, relative to the diameter for the catheter or the like, one would utilize the smaller and precise passage for the drilling, and then utilize the larger, potentially less precise (although it might not be less precise, and could be more in fact) catheter, etc.

In an exemplary embodiment, there is a method as detailed herein where, for example, after removing the material in the second state from contact with the wall of the outer ear, executing a minimally invasive surgical action to at least reach a location proximate an outer barrier of an inner ear of the live human relying at least in part on the material in the second state and/or in a third state more stiff than that which is the case in the second state or a secondary product based on the material in the second state and/or in the third state or a fourth state more stiff than that which is the case in the second state, wherein the action of executing a minimally invasive surgical action includes relying on visual feedback based at least in part on an artificial device (an endoscope, or using a laser pointer, etc.) while moving a working component towards a target location on a barrier between a middle ear and an inner ear of the human, wherein the artificial device extends through a passage through the material in the second state and/or in the third state or a passage through the product. In accordance with the teachings above, there can be a second passage where, for example, a drill bit or the like extends therethrough.

And it is noted that in at least some exemplary embodiments, the passages do not necessarily require the cross-sections to be circular. This can be the case for the irrigation fluid, where the tube or otherwise hose for the irrigation can come in any shape channel. Moreover, a CMOS camera chip with two light fibers next to such can sit in a jellybean cross-section shaped channel wrapped around the circular working channel in the center by way of example.

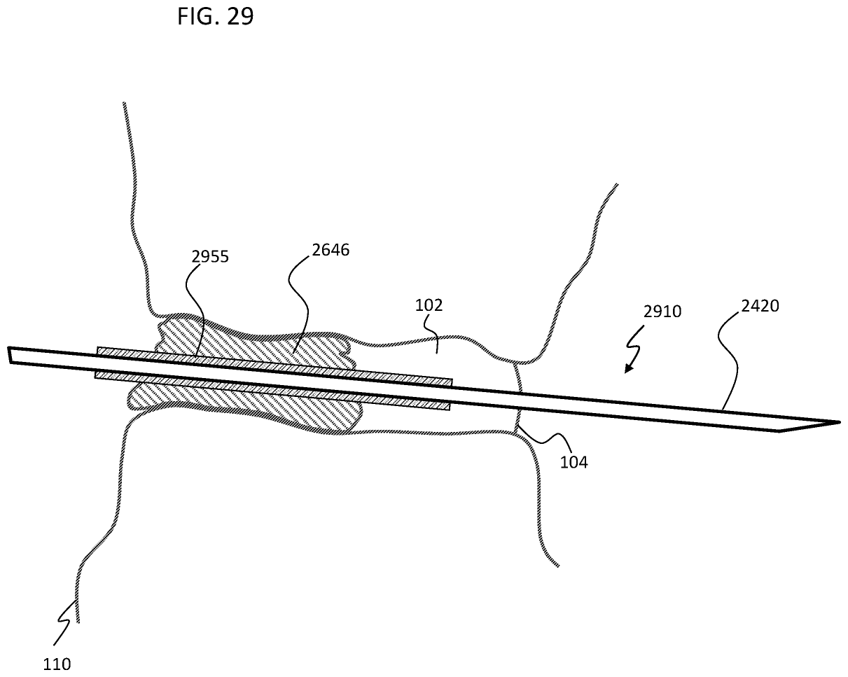

In an exemplary embodiment, the apparatus is configured to enable the hollow medical component to move in an axial direction relative to the support structure. This can be a nature of the material of the support structure or can be a feature of the componentry that interface with the support structure. By way of example, FIG. 29 presents an exemplary device 2910 that has a termination 2420 supported by a bushing 2955, about which the support structure 2646 has been molded. The bushing 2955 is sized and dimensioned to enable the termination 2420 to move in the axial direction, but the tolerancing between the bushing and the outer diameter of the termination 2420 are such that the movement in the radial direction is prevented. And in this regard, the bushing 2955 can be combined with the shroud device detailed above—the shroud could go around the bushing which goes around the termination 2420, and the shroud could have both an outboard shroud and an inboard shroud, where the latter establishes a barrier between the material and the bushing (or the termination—indeed, the shroud concept can also serve as a bushing in fact—the outboard shroud can have the orifices of the like, and the inboard shroud can be solid to prevent the material from reaching the termination 2420, where the inboard shroud (which can be a tube or a hollow cylinder) is sized and dimensioned to permit the termination to slide therein to enable radial movement but prevent the movement in the axial direction.

And it is noted that in an alternate embodiment, the apparatus is configured to prevent the hollow medical component from moving in an axial direction relative to the support structure. This can be as a result of a “tight” interface between the support structure and the medical component. This could be via the use of an additional material such as an adhesive or the like. Any arrangement device and/or system that can enable the apparatus to prevent the hollow medical component from moving in the axial direction relative to the support structure can be utilized in at least some exemplary embodiments.

In an exemplary embodiment, the support structure enables fixation of position of a longitudinal axis of the hollow medical component in the radial direction. In an exemplary embodiment, the support structure enables fixable position of a longitudinal axis of the hollow medical component in an X and Y direction, and in respective planes normal to the X and Y direction (where the Z direction is the long axis of the hollow component).

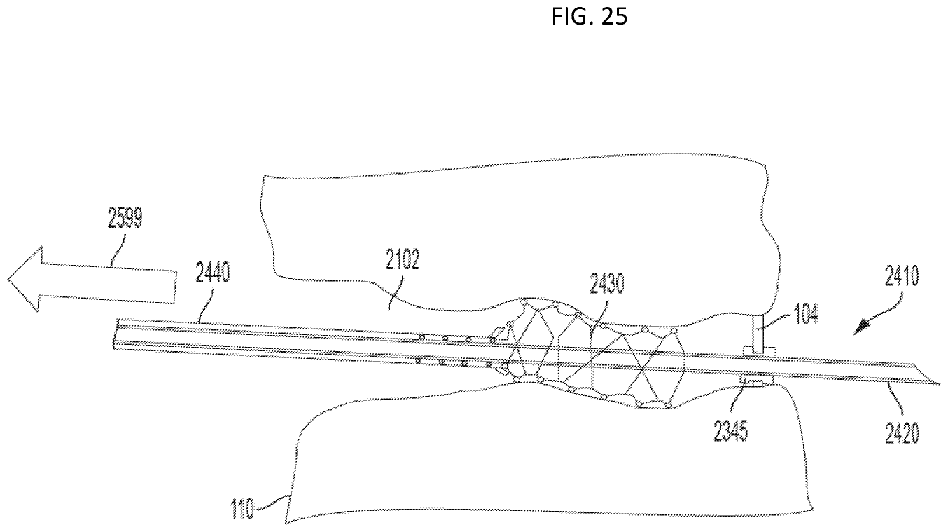

Consistent with the embodiment of FIG. 26, in an exemplary embodiment, the apparatus can further include a flow channel fluidically isolated from the hollow portion of the hollow medical component (the conduit of the cannula, for example), the flow channel being configured to channel a flowing substance (material in the first state, for example) to locations outboard of the hollow medical component to establish the support structure, the flowing substance being a substance that, upon a change of state from that in which it was during flowing, enables no support structure to fix the trajectory of the medical component. Consistent with the embodiment of FIGS. 24 and 25, for example, in an exemplary embodiment, the support structure is an extendable stent/an uncollapsible stent. In an exemplary embodiment, the support structure is a form-in-place elongated seal (or plug with a hole therethrough).

Also, consistent with the teachings detailed above, in an exemplary embodiment, the apparatus is located in the ear canal in an anatomically correct manner and the hollow medical component extends past the support structure, through a tympanic membrane of the human, such that a distal end of the hollow component is located proximate a barrier between the middle ear and an inner ear of the person.

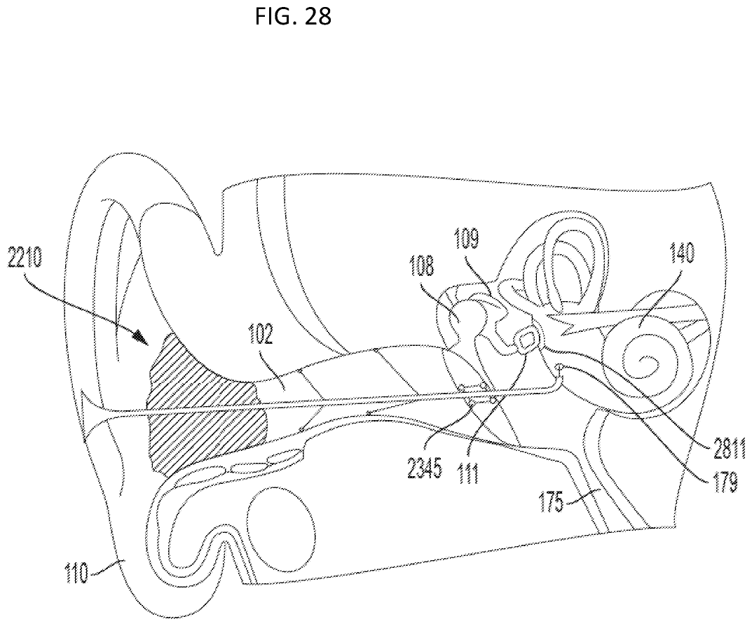

In an exemplary embodiment, the hollow medical component extends along a first axis for most of its length, and then, at a distal portion thereof, extends in a direction away from the first axis. This can be seen in FIG. 28 by way of example, where the device 2810 includes the curved distal portion 2820 of the hollow medical component. This can have utilitarian value with respect to reaching the round window niche 179 (the oval window 2811 is shown for reference). This can have utilitarian value in a scenario where, for example, the target area cannot be approached at a desired angle (e.g. perpendicular) in a straight line through the ear canal, and thus the utility of the cannula's/termination's axis not being straight but instead bending at the distal end (and this can occur at one or more locations—there can be multiple changes of direction). The cannula might be bent prior to insertion, or, in other embodiments, the cannula is inserted as a straight tube first and an internal structure, such as a steerable guide wire or flexible and steerable endoscope (or both) adjusts the shape of the cannula (and this can be done after the tip enters the middle ear cavity for example. The form giving member can, in some embodiments, remain in the cannula for subsequent surgical steps and/or the cannula can become rigid to memorize the shape so that the shape forming member (steerable guide wire) can be withdrawn. This shape memory can be permanent or can be revertible to allow more easy withdrawal of the guide cannula at the completion of the procedure, for example.

In an exemplary embodiment, a catheter or some other delivery device includes a steerable tip that is configured to be steered at angles between 1 and 130 degrees or any value or range of values therebetween in 1° increments degrees.

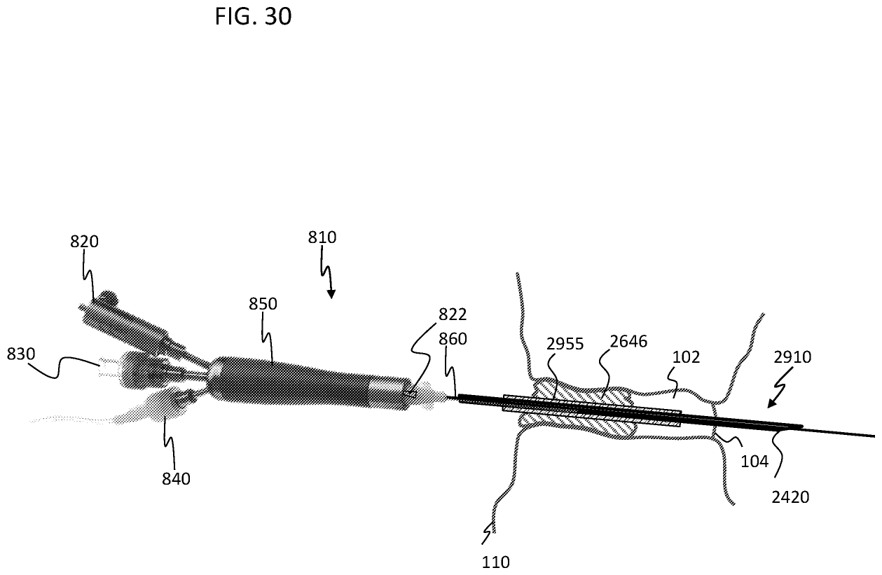

FIG. 30 presents an exemplary embodiment of an ear system endoscope 810 in use with an exemplary custom ear canal guide according to an exemplary embodiment, here, the custom ear canal guide of the embodiment of FIG. 29 just detailed which has the combined tissue interface body 2646 and the cannula guide 2420. It is noted that in an exemplary embodiment, the arrangement presented in FIG. 30 is representative of the utilization of the endoscope 810 with the embodiments detailed above with respect to the custom ear canal guide without the canola guide, such as the embodiment of FIG. 19 above. The ear system endoscope can be configured to incise through tissue to reach duct(s) of an inner ear of a human and to deliver therapeutic substance to the duct(s) through a resulting incision, but can also be used with the teachings herein, and thus need not necessarily have the ability to incise. Briefly, the endoscope 810 can be used to deliver a therapeutic substance into the duct of the cochlea (whether through a cochleostomy, or by puncturing the round window or the oval window, etc.), with, for example, the cannula guide used to guide the termination 860 of the endoscope 810 (and in other embodiments, instead of a cannula guide, a termination of the ear canal guide is used to deliver the therapeutic substance) and thus embodiments include methods of utilizing the ear system endoscope 810 to provide therapeutic substance into the ducts of the cochlea from a location outside the middle ear, and from outside the outer ear for that matter in some embodiments, through the middle ear, and into the inner ear, utilizing the cannula guides detailed herein. Briefly, in an exemplary embodiment, the therapeutic substance on a molecular basis moves from the location outside the middle ear and/or from outside the outer ear, to the inner ear, within 1 minute, within 45, 30, 25, 20, 15, 10, or 5 seconds or less. This as opposed to, for example, that which may occur utilizing diffusion or the like where the therapeutic substances simply provided to the round window niche, and the therapeutic substance diffuses through the round window into the cochlea.

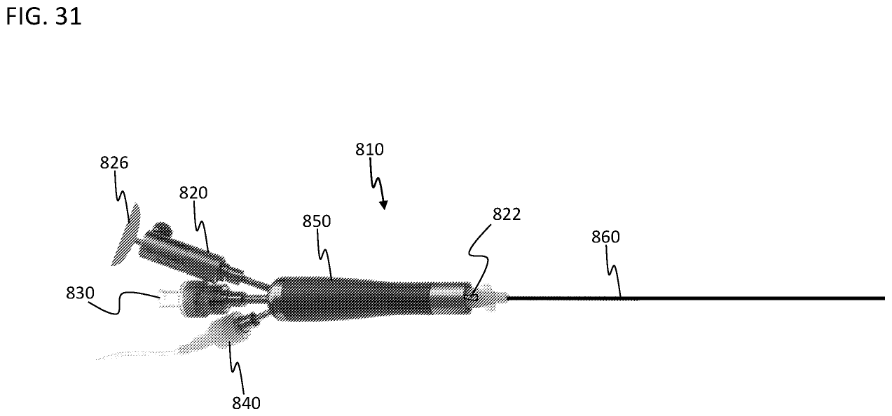

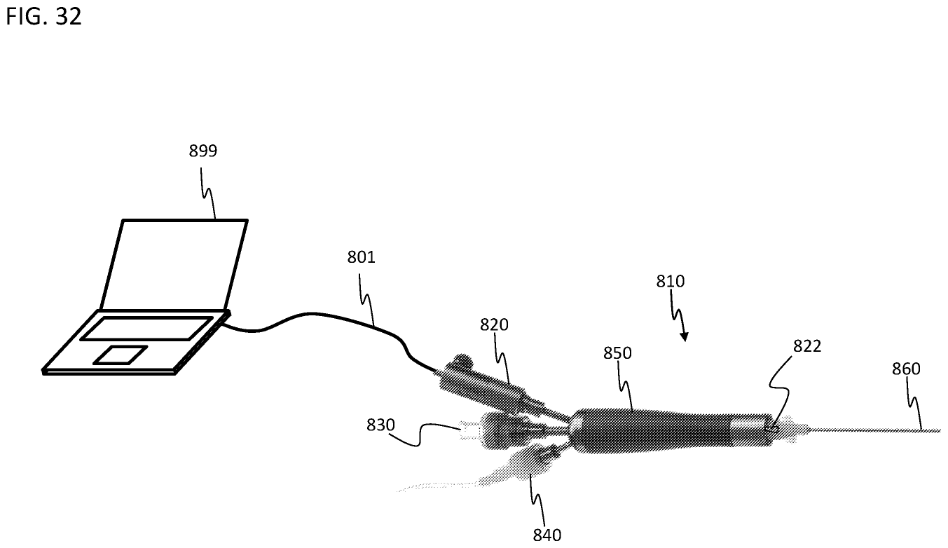

Returning back to FIG. 30, the ear system endoscope 810 includes various components, such as, an optical channel 820 which enables data indicative of an image inside the ear system to be conveyed to a location outside the ear system. This can be based on fiber optics or wired communication. Corollary to this is that there is a camera 822 or some other light capture arrangement (a purely optical device can be used, which may magnify light captured at the working end) that is part of the ear system endoscope, that is in electrical communication or light communication with the optical channel 820. The optical channel 820 can be in communication with a display 826 (see FIG. 31) or some other image conveying device, such as a lens (again, a purely optical system can be used, akin to the output of a traditional microscope, for example). Alternatively, and/or in addition to this, the optical channel 820 can be configured to provide the data indicative of an image via a cable 801 (or a wireless link, such as a Blue Tooth link) to a “remote” monitor or the like positioned away from the ear system endoscope 810, such as a monitor of a laptop see FIG. 32 and laptop 899 connected to output cable 801 from optical channel 820 or a desktop computer, which could be located in the same room in which the ear system endoscope is being utilized to reach the cochlea. Accordingly, it can be seen that in some scenarios of use the surgeon or other healthcare professional or whoever is utilizing the ear system endoscope guides the endoscope through the cannula guide to the target by looking at the endoscope/looking in the direction of the side of the human's head/looking at the outer ear, while in some other embodiments, the guiding is executed while the user is looking at a computer screen and thus looking in a direction away from the human's head/outer ear, etc.

Again referring back to FIG. 30, the ear system endoscope 810 further includes a surgical tool port 830. This port can be configured to receive a needle and/or a drill and/or a laser generator and/or output and/or or a micro tweezers or can just be a general port to which a therapeutic substance reservoir or a therapeutic substance supply line can be attached to deliver therapeutic substance to the inner ear. Furthermore, in an example, the port can receive a biopsy tool and/or blades and/or forceps and/or microneedles (microneedle array/assembly), catheters, etc. Moreover, by way of example only and not by way of limitation, in an exemplary embodiment, tools that are configured to take a sample of a fluid or a liquid within the body, such as perilymph within the cochlea and/or the fluid within the semicircular canals, example, can be extended through the aforementioned port(s).

An exemplary embodiment includes a steerable tip endoscope with a channel for vision and a channel for tools (e.g., a drill tip and/or a therapeutic substance delivery tool (e.g., a conduit extends through a channel), the tool bending in compliance with the tip angle. Thus, there could be 2, 3, 4 or more surgical tool ports 830.

And in this regard, by way of example only and not by way of limitation, a cannula guide which is straight can be utilized, and providing that there is sufficient offset from the target, the distal portion of the endoscope can be steered from the distal end of the cannula guide to the target (thus achieving the functionality of, for example, the device of FIG. 28).

Also, as can be seen, there is an irrigation port 840, which can be utilized to provide irrigation fluid, such as a saline liquid, to the working end of the ear system endoscope, which can be used to provide irrigation of the middle and/or inner ear during use of the tool. The tympanic membrane can also be irrigated utilizing the irrigation features of the ear system endoscope in some embodiments.

As can be seen, the channel 820 and the ports 830 and 840 are supported by a body 850, which can be ergonomically designed so that a surgeon or other healthcare professional and easily grip and support the ear system endoscope with a thumb and one or more fingers of a hand, or the entire hand.

The working end of the ear system endoscope 810 includes a termination 860, which can be a tube made of metal, such as stainless steel (e.g., 316), or some other material. In an exemplary embodiment, the termination 860 can correspond to those of at least the body portion (which may or may not include the sharp end) of a syringe termination approved for use in the United States as of Jun. 2, 2021. This can be a low volume, medium volume, or high volume termination. In an exemplary embodiment, the termination is sized and dimensioned the termination can extend from outside the ear canal, or at least outside the tympanic membrane 104, through the cannula guide, to the promontory (and into the promontory) and/or to the round window niche of the cochlea.

Embodiments include utilizing any one or more of the above endoscopes or devices disclosed herein with the various custom ear canal guides disclosed herein, and thus there are methods of such and systems that include both devices.

It is briefly noted that any reference to an endoscope and/or a drill bit and/or a hand tool herein corresponds to a disclosure of an alternate embodiment that includes that feature in a more generic tool and/or in another of the tools disclosed herein, and visa-versa.

It is noted that any disclosure of a device and/or system herein corresponds to a disclosure of a method of utilizing such device and/or system. It is further noted that any disclosure of a device and/or system herein corresponds to a disclosure of a method of manufacturing such device and/or system. It is further noted that any disclosure of a method action detailed herein corresponds to a disclosure of a device and/or system for executing that method action/a device and/or system having such functionality corresponding to the method action. It is also noted that any disclosure of a functionality of a device herein corresponds to a method including a method action corresponding to such functionality. Also, any disclosure of any manufacturing methods detailed herein corresponds to a disclosure of a device and/or system resulting from such manufacturing methods and/or a disclosure of a method of utilizing the resulting device and/or system.

Unless otherwise specified or otherwise not enabled by the art, any one or more teachings detailed herein with respect to one embodiment can be combined with one or more teachings of any other teaching detailed herein with respect to other embodiments, and this includes the duplication or repetition of any given teaching of one component with any like component. Also, embodiments include devices systems and/or methods that explicitly exclude any one or more of a given teaching herein. That is, at least some embodiments include devices systems and/or methods that explicitly do not have one or more of the things that are disclosed herein.

While various embodiments of the present invention have been described above, it should be understood that they have been presented by way of example only, and not limitation. It will be apparent to persons skilled in the relevant art that various changes in form and detail can be made therein without departing from the scope of the invention.

1. A method, comprising:
accessing an ear system of a live human;
transporting a material in a first state so that the material comes into contact with a wall of the outer ear;
at least permitting the first material to transform to a second state; and
removing the material in the second state from contact with the wall of the outer ear, wherein the material in the second state retains a memory of a shape of the wall of the outer ear into which the material was in contact when transforming to the second state.

2. The method of claim 1, wherein:
the material in the second state and/or in a third state more stiff than that which is the case in the second state establishes at least a portion of a unique embryonic surgical guide configured for unique steady access to structure of an ear of the live human.

3. The method of claim 1, wherein:
the material in the second state retains a memory of a shape of a barrier between a middle ear and an inner ear of the live human into which the material was in contact when transforming to the second state.

4-5. (canceled)

6. The method of claim 1, further comprising:
after removing the material in the second state from contact with the wall of the outer ear, executing a minimally invasive surgical action to at least reach a location proximate an outer barrier of an inner ear of the live human relying at least in part on the material in the second state and/or in a third state more stiff than that which is the case in the second state or a secondary product based on the material in the second state and/or in the third state or a fourth state more stiff than that which is the case in the second state.

7. The method of claim 1, wherein:
the action of transporting the material in the first state includes transporting the material so that the material comes into contact with a barrier between the middle ear and the inner ear; and
the material in the second state retains a memory of a shape of an outer surface of the barrier between the middle ear and the inner ear.

8. The method of claim 7, wherein:
the action of removing the material in the second state includes pulling a portion of the material that interfaced with the barrier between the middle ear and the inner ear through an incision in a tympanic membrane, a relaxed maximum diameter of the incision being smaller than a maximum diameter of the portion in a relaxed state that is compressed to fit through the incision.

9-11. (canceled)

12. An apparatus, comprising:
a negative model of an inner wall surface of an ear canal of an outer ear unique to a specific person, which model has outer contours to the surface of the actual wall of the ear canal of the specific person.

13. The apparatus of claim 12, wherein:
the negative model includes a passage extending from a distal end to a proximal end of the model, the passage having a longitudinal axis that would bisect a barrier between the middle ear and the inner ear if the model was located in an anatomically correct manner in the outer ear.

14. The apparatus of claim 12, wherein:
the negative model includes a passage extending from a distal end to a proximal end of the model, the passage having a diameter normal to a longitudinal axis of the passage that is between 0.5 mm to 8 mm.

15. The apparatus of claim 12, wherein:
the negative model includes a passage extending from a distal end to a proximal end of the model, the passage having a longitudinal axis that would bisect a barrier between the middle ear and the inner ear if the model was located in an anatomically correct manner in the outer ear, the bisection being at a location below a round window niche of the person, the location below being relative to an orientation where an oval window of the person is above the round window.

16. The apparatus of claim 12, wherein:
the apparatus is located in the ear canal in an anatomically correct manner; and
a termination or a drill bit extends through the model and through a tympanic membrane of the person, such that a distal end of the termination or the drill bit is located proximate a barrier between the middle ear and an inner ear of the person.

17. The apparatus of claim 12, wherein:
the apparatus is located in the ear canal in an anatomically correct manner; and
a drill bit extends through the model, through a tympanic membrane of the person, such that a distal end of the drill bit is located proximate a barrier between the middle ear and an inner ear of the person.

18. The apparatus of claim 12, wherein:
the apparatus further includes a negative model of a portion of a barrier between a middle ear and an inner ear of the specific person.

19-27. (canceled)

28. An apparatus, comprising:
a hollow medical component; and
a support structure distinct from the hollow medical component and supporting the hollow medical component, the support structure being configured to interface with an interior of a unique ear canal of a specific human and support the hollow medical component in a fixed trajectory relative to a target in a middle ear of the specific human.

29. The apparatus of claim 28, further comprising:
a flow channel fluidically isolated from the hollow portion of the hollow medical component, the flow channel being configured to channel a flowing substance to locations outboard of the hollow medical component to establish the support structure, the flowing substance being a substance that, upon a change of state from that in which it was during flowing, enables the support structure to fix the trajectory of the medical component.

30. The apparatus of claim 28, wherein:
the support structure is an uncollapsible stable stent.

31. The apparatus of claim 28, wherein:
the support structure is a form-in-place elongated seal.

32. The apparatus of claim 28, wherein:
the hollow medical component extends along a first axis for most of its length, and then, at a distal portion thereof, extends in a direction away from the first axis.

33. The apparatus of claim 28, wherein:
the hollow medical component is a surgical guide or a termination.

34. The apparatus of claim 28, wherein:
the apparatus is configured to enable the hollow medical component to move in an axial direction relative to the support structure.

35-39. (canceled)
